(19) United States
(12) Patent Application Publication
(10) Pub. No.: US 2005/0221326 A1
(43) Pub. Date: Oct. 6, 2005

(54) Oligonucleotides antibodies and kits including same for treating prostate cancer and determining predisposition thereto

(76) Inventors: Avi Orr-Urtreger, Kiryat Ono (IL); Hanna Rennert, Zikhron Yaakov (IL); Dani Bercovich, Doar Na Darom HaGolan (IL); Anat Bar-Shira, Tel Aviv (IL); Yuval Yaron, Ramat Gan (IL)

Correspondence Address: Martin Moynihan;Anthony Castorina, Suite 207, 2001 Jefferson Davis Highway, Arlington, VA, 22202, US

(21) Appl. No.: 10/516,303

(22) PCT Filed: Jun. 12, 2003
(86) PCT No.: PCT/IL03/00507

§ 371 Date: Dec. 10, 2004

Related U.S. Application Data
(60) Provisional application No. 60/387,589, filed on Jun. 12, 2002.

(51) Int. Cl. C12Q 1/68

(52) U.S. Cl. 435/6

(57) ABSTRACT

A method of determining predisposition of an individual of Ashkenazi descent to prostate cancer is provided. The method comprises determining a presence or absence of at least one nucleic acid sequence alteration in at least one allele of a RNASEL gene of the individual, wherein the presence of the at least one nucleic acid sequence alteration indicates predisposition to prostate cancer in the individual.

### FIELD AND BACKGROUND OF THE INVENTION

The present invention relates to oligonucleotides, antibodies and kits including same, which can be used to treat prostate cancer and determine predisposition thereto. More particularly, the present invention can be used to determine predisposition of individuals to prostate cancer especially those of Ashkenazi descent.

Prostate cancer is the most common cancer in men in many western countries, and the second leading cause of cancer deaths in this population. It accounts for more than 40,000 deaths in the United States annually. The number of deaths is likely to continue rising over the next 10 to 15 years. In the United States, prostate cancer is estimated to cost $1.5 billion per year in direct medical expenses. In addition to the burden of suffering, it is a major public-health issue.

Most prostate cancers are adenocarcinomas, cancers that arise in glandular cells of the prostate epithelial tissue, although other types of prostate cancer also exist including, transitional cell carcinomas, ductal carcinomas, and squamous cell carcinomas. Prostate cancers usually progress slowly and produce no symptoms in the initial stages. Gradually, the tumor may enlarge the prostate gland, pressing on the urethra and causing painful or frequent urination and blood in the urine or semen. Sometimes pain in the lower back, pelvis, or upper thighs may signal that prostate cancer cells have spread to the ribs, pelvis, and other bones. The ability of the tumor cells to spread to distant organs is the main cause of prostate cancer-related deaths. The regional lymph nodes and the bone system are the main sites of metastasis.

Prostate cancer is considered a disease of old age since it does not typically appear until the sixth decade [Kumar (1997) in Basic Pathology, 6^{th} ed, pp 584-588, W. B. Saunders Co., Philadelphia]. In addition to aging, hormonal, environmental and genetic factors are all believed to play roles in pathogenesis of prostate cancer.

Genetic studies have demonstrated that the lifetime risk for the development of prostate cancer increases 2-3 fold in men with a single first-degree relative with prostate cancer [Steinberg (1990) Prostate 17:337-347]. If both first and second degree relatives are affected, the risk may be about 8-fold higher than in man without such a family history. Additionally, men with a family history of prostate cancer are likely to develop the disease at younger age. Hereditary genetic factors in prostate cancer are believed to be as significant as in colon and breast cancers [Lichtenstein (2000) N. Eng. J. Med. 343:78-85; Verkasalo (1999) 83:743-9].

Segregation analysis supports rare autosomal dominant, highly penetrant HPC genes in hereditary prostate cancer with early onset [Carter (1992) Proc. Natl. Acad. Sci. USA 89:3367-3371]. The HPC genes are considered to account for about 43% of early onset (<55) disease and 9% of all cases of prostate cancer.

Based on family-based linkage analysis, eight putative prostate cancer susceptibility loci have been reported (MIM 176807) [Smith, (1996) Science 274:1371-1374; Berthon, (1998) Am. J. Hum. Genet. 62:1416-1424; Xu, (1998) Nat Genet. 11:198-200; Berry, (2000) Am. J. Hum. Genet. 67:82-91], from which, only the BPC2/ELAC2 (GenBank Accession No: MIM 605367) at 17p11 and HPC1/RNASEL (GenBank Accession No: MIM 180435) at 1q25 have been cloned [Tavtigian, (2001) Nat. Genet. 27:172-180; Carpten, (2002) Nat. Genet. 30:181-184].

Notably, germline mutations of the ELAC2 gene are rare in hereditary prostate cancer (HPC) [Tavtigian, (2001) supra; Wang, (2001) Cancer Res. 61: 6494-6499]. In the RNASEL gene, on the other hand, a truncating mutation (E265X), was detected in a single HPC1-linked family [Carpten, (2002) Nat. Genet. 30:181-184], and was also associated with early-onset prostate cancer in Finish families with HPC [Rokman, (2002) Am. J. Genet. 70:1299-1304]. In addition, an R462Q mutation in RNASEL was shown to be associated with familial prostate cancer incidence, and with more advanced disease state, substantiating RNASEL as a prostate cancer predisposition gene.

HPC1/RNASEL encodes the 2′,5′-oligoisoadenylate-synthetase-dependent ribonuclease L, which regulates cell proliferation and apoptosis through the interferon-regulated 2-5A pathway [Hassel, (1993) EMBO J 12:3297-3304; Zhou, (1993) Cell 72:753-765; Bisbal, (1995) J Biol Chem 270:13308-13317], and it has been suggested to be a tumor suppressor gene [Carpten, 2002 supra]. RNASEL controls the balance between hormonally regulated growth and cell death in the prostate [Carpten (2002) Nat. Genet. 30:181-184]. Accumulative evidence suggest a tumor suppressive role for RNASEL including involvement in the antiproliferative activity of interferon, location of RNASEL at chromosome 1q25, a region deleted or rearranged in some breast cancers [Hassel (1993) EMBO J. 12:3297-3304; Squire (1994) Genomics 19:174-175; Lengyel (1993) Proc. Natl. Acad. Sci. USA 90:5893-5895].

Unlike BRCA1/2 in breast cancer [Struewing, (1995) Nat. Genet. 11:198-200] and APC I1307K in colon cancer [Laken, (1997) Nat. Genet. 17:79-83], very little is known about susceptibility genes for prostate cancer in Jewish men especially of Ashkenazi descent which together with Finns and Icelanders represent the only ethnic groups with clusters of mutations known to date.

While reducing the present invention to practice, the present inventors uncovered novel mutations in the RNASEL gene, which are associated with development of prostate cancer. These findings can be used to treat prostate cancer and determine predisposition thereto in numerous individuals world wide especially those with Ashkenazi descent.

### SUMMARY OF THE INVENTION

According to one aspect of the present invention there is provided a method of determining predisposition of an individual of Ashkenazi descent to prostate cancer, the method comprising determining a presence or absence of at least one nucleic acid sequence alteration in at least one allele of a RNASEL gene of the individual, wherein the presence of the at least one nucleic acid sequence alteration indicates predisposition to prostate cancer in the individual.

According to further features in preferred embodiments of the invention described below, the at least one nucleic acid sequence alteration is selected from the group consisting of:

(i) a deletion spanning nucleotides 471474 of SEQ ID NO: 1;

(ii) a C to T substitution at nucleotide 354 of SEQ ID NO: 1; and

(iii) a deletion at nucleotide 11338427 of SEQ ID NO: 2.

According to another aspect of the present invention there is provided a method of determining predisposition of a subject to prostate cancer, the method comprising determining a presence or absence of at least one nucleic acid sequence alteration in at least one allele of a RNASEL gene of an individual, the at least one nucleic acid sequence alteration being selected from the group consisting of:

(i) a deletion spanning nucleotides 471-474 of SEQ ID NO: 1;

(ii) a C to T substitution at nucleotide 354 of SEQ ID NO: 1; and

(iii) a deletion at nucleotide 11338427 of SEQ ID NO: 2,

wherein the presence of the at least one nucleic acid sequence alteration indicates predisposition to prostate cancer in the individual.

According to yet another aspect of the present invention there is provided an oligonucleotide specifically hybridizable with a nucleic acid sequence alteration selected from the group consisting of:

(i) a deletion spanning nucleotides 471474 of SEQ ID NO: 1;

(ii) a C to T substitution at nucleotide 354 of SEQ ID NO: 1; and

(iii) a deletion at nucleotide 11338427 of SEQ ID NO: 2.

According to still further features in the described preferred embodiments the oligonucleotide is hybridizable with SEQ ID NO: 1 under hybridization conditions of hybridization solution containing 10% dextran sulfate, 1 M NaCl, 1% SDS and 5×10⁶ cpm ³²p labeled probe, at 65° C., with a final wash solution of 1×SSC and 0.1% SDS and final wash at 50° C.

According to still further features in the described preferred embodiments the oligonucleotide includes at least 10 nucleotides and no more than 50 nucleotides.

According to still another aspect of the present invention there is provided a kit for diagnosing prostate cancer or a predisposition to prostate cancer in a subject, the kit comprising the oligonucleotide of claim 7 and at least one reagent for detecting hybridization of the oligonucleotide with a nucleic acid sequence isolated from the subject.

According to still further features in the described preferred embodiments the at least one reagent is selected suitable for detecting hybridization via an assay selected from the group consisting of PCR, RT-PCR, chip hybridization, RNase protection, in-situ hybridization, primer extension, Southern blot, Northern blot and dot blot analysis.

According to an additional aspect of the present invention there is provided a method of treating a subject having, or being predisposed to, prostate cancer, the method comprising specifically downregulating in the subject expression of a mutated RNASEL transcript having at least one sequence alteration selected from the group consisting of:

(i) a deletion spanning nucleotides 471-474 of SEQ ID NO: 1;

(ii) a C to T substitution at nucleotide 354 of SEQ ID NO: 1; and

(iii) a deletion at nucleotide 11338427 of SEQ ID NO: 2, thereby preventing the formation, or halting the progression of, prostate cancer in the subject.

According to still further features in the described preferred embodiments specifically downregulating expression of the mutated RNASEL in the subject is effected by administering to the subject an oligonucleotide capable of specifically inactivating the mutated RNASEL transcripts.

According to still further features in the described preferred embodiments the oligonucleotide is a single or double stranded polynucleotide.

According to still further features in the described preferred embodiments the oligonucleotide is at least 10 nucleotides long.

According to still further features in the described preferred embodiments the oligonucleotide is hybridizable in either sense or antisense orientation.

According to yet an additional aspect of the present invention there is provided a method of determining sensitivity of a subject to prospective interferon therapy, the method comprises determining a presence or absence of at least one nucleic acid sequence alteration selected from the group consisting of a deletion spanning nucleotides 471-474 of SEQ ID NO: 1 and/or a deletion at nucleotide 11338427 of SEQ ID NO: 2, wherein the presence of the at least one sequence alteration indicates poor sensitivity of the subject to the prospective interferon therapy.

According to still further features in the described preferred embodiments the presence or absence of the at least one nucleic acid sequence alteration is determined in samples isolated from blood, amniotic fluid, or chorionic villi.

According to still further features in the described preferred embodiments determining the presence or absence of the at least one nucleic acid sequence alteration is effected by the use of oligonucleotide hybridization.

According to still further features in the described preferred embodiments determining the presence or absence of the at least one nucleic acid sequence alteration is effected by an assay selected from the group consisting of PCR, DNA sequencing and SSCP analysis.

According to still an additional aspect of the present invention there is provided an antibody or antibody fragment being capable of specifically binding at least a portion of amino acid residues 1-55 of an RNASEL polypeptide.

According to a further aspect of the present invention there is provided an antibody or antibody fragment being capable of specifically binding a polypeptide including an amino acid sequence set forth in SEQ ID NO: 41.

According to still further features in the described preferred embodiments the antibody or antibody fragment is directed at the amino acid sequence set forth in SEQ ID NO: 41.

According to yet a further aspect of the present invention there is provided a kit for diagnosing prostate cancer or a predisposition to prostate cancer in a subject, the kit comprising the antibody or antibody fragment and at least one reagent for detecting binding of the antibody or antibody fragment to a polypeptide isolated from the subject.

According to still further features in the described preferred embodiments detecting binding of the antibody or antibody fragment to the polypeptide is effected by an assay selected from the group consisting of immunohistochemistry, ELISA, RIA, Western blot analysis, FACS analysis, an immunofluorescence assay, and a light emission immunoassay.

According to still further features in the described preferred embodiments the antibody or antibody fragment is coupled to an enzyme.

According to still further features in the described preferred embodiments the antibody or antibody fragment is coupled to a detectable moiety selected from the group consisting of a chromogenic moiety, a fluorogenic moiety, a radioactive moiety and a light-emitting moiety.

According to still a further aspect of the present invention there is provided a method of determining predisposition of a subject to prostate cancer, the method comprising determining a presence or absence of at least one amino acid sequence alteration in an RNASEL polypeptide of an individual, the at least one amino acid sequence alteration being a translation product of:

(i) a deletion spanning nucleotides 471-474 of SEQ ID NO: 1; or

(ii) a deletion at nucleotide 11338427 of SEQ ID NO: 2,

wherein the presence of the at least one amino acid sequence alteration indicates predisposition to prostate cancer in the individual.

The present invention successfully addresses the shortcomings of the presently known configurations by providing oligonucleotides, antibodies and kits including same, which can be used to treat prostate cancer and determine predisposition thereto.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1a-c are graphs depicting the 471delAAAG frameshift mutation in RNASEL exon 1, as determined by sequencing. FIG. 1a depicts a normal control; FIG. 1b depicts a PRCA affected individual with heterozygous germline mutation; FIG. 1c depicts LOH for 471 delAAAG mutation in a dissected tumor from a PRCA affected carrier. Note that the deletion start point and the AAAG deleted sequence are indicated by an arrow and a box, respectively.

FIGS. 2a-d are graphs depicting a DHPLC analysis of 471delAAAG frameshift mutation in RNASEL using a PCR product of exon 1 as in FIGS. 1a-c. FIG. 2a depicts a normal control; FIG. 2b depicts a 471delAAAG hetroduplex from an heterozygous PRCA patient; FIG. 2c depicts a 471de1AAAG homoduplex from an homozygous PRCA patient; FIG. 2d depicts a 354C>T hetroduplex.

FIGS. 3a-d are graphs depicting the IVS5+1delG splice site mutation in RNASEL exon-intron 5 by DHPLC (FIGS. 3a-b) and sequencing (FIGS. 3c-d).

### DESCRIPTION OF THE PREFERRED EMBODIMENTS

The present invention is of oligonucleotides, antibodies and kits including same, which can be used to determine predisposition and treat prostate cancer particularly in individuals of Ashkenazi descent.

The principles and operation of the present invention may be better understood with reference to the drawings and accompanying descriptions.

Before explaining at least one embodiment of the invention in detail, it is to be understood that the invention is. not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Prostate cancer has evolved as a major health problem in the male population of the Western world. It is the most commonly diagnosed malignancy and the second leading cause of cancer death, representing nearly 29% of all male cancer deaths. In the year 2001, about 200,000 new cases of prostatic adenocarcinomas were projected in the United States. Prostate cancer is usually an indolent disease, though 25-30% of the tumors become metastatic, resulting in almost 40,000 deaths annually [Parker (1996) CA Cancer J. Clin. 46:5-27].

Segregation analysis of familial prostate cancer suggests an autosomal dominant node of inheritance which accounts for 9% of the total prostate cancer occurrence by age 85 years [Carter (1993) J. Urol. 150:797-802; Gronberg (1997) 146:552-7; Schaid (1998) 62:1425-38].

To date, at least six different chromosomal regions have been postulated to contain prostate cancer predisposition genes. Recently, researchers have focused on two particular genes, HPC2 (hereditary prostate cancer 2), and RNASEL, also called HPC1 which encodes the 2′,5′-oligoisoadenylate-synthase-dependent ribonuclease L which regulates cell proliferation and apoptosis through the interferon-regulated 2-5A pathway.

While HPC2 mutations have only been identified in 3 families with hereditary prostate cancer so far, in the RNASEL gene at least two mutations (i.e., E265X and R462Q) have been found more frequently in families with hereditary prostate cancer than in cancer-free controls, substantiating RNASEL as a prostate cancer predisposition gene.

Unlike breast cancer genetic studies which indicate significantly higher frequency of BRCA1 and BRCA2 mutations in Ashkenazi Jews as compared to the general population, to date very little is known about susceptibility genes for prostate cancer in Jewish man particularly those of Ashkenazi descent.

While reducing the present invention to practice, the present inventors uncovered novel mutations in RNASEL, which are associated with prostate cancer in Ashkenazi Jews.

As is illustrated in the Examples section which follows, the present inventors found through laborious screening and experimentation, a novel RNASEL frameshift founder mutation, 471delAAAG, and an allelic silent variant 354C>T in 4.0% and 3.8% of Ashkenazi Jews, respectively; these mutations were not detected in non-Ashkenazi Jews tested. The frequency of the 471delAAAG mutation among prostate cancer patients (7.9%) was significantly higher than in elderly controls (1.5%, P≦0.025). 28% of patients with a first degree prostate cancer relative were carriers of this mutation. Homozygous and heterozygous 471delAAAG mutations were detected in two brothers affected with prostate cancer, and loss of heterozygocity was further demonstrated in the dissected tumor obtained from the heterozygous sibling. In contrast to the frameshift mutation, the frequency of 354C>T was similar among prostate cancer patients and controls, however all patients carrying this mutation had a family history of cancer. All 471delAAAG mutation carriers shared the same haplotype based on flanking dinucleotide markers, as compared to 32.5% of non-carrier prostate cancer patients (P≦0.001), suggesting a founder effect. An additional allelic variant in RNASEL in exon-intron 5, IVS5+1delG, a donor splice site change, was identified in an Ashkenazi prostate cancer patient, which is likely to cause a weak allele, further supporting the role of RNASEL in prostate cancer pathogenesis.

These findings allow for the first time to treat and determine predisposition to prostate cancer in subjects of Ashkenazi descent in which genetic factors appear to play a critical role in cancer development.

Thus, according to one aspect of the present invention, there is provided a method of determining predisposition of an individual of Ashkenazi descent to prostate cancer.

As used herein the term “predisposition” refers to a latent susceptibility to prostate cancer, which may lead, under certain conditions, to the formation of prostate cancer.

As used herein the phrase “prostate cancer” refers to adenocarcinomas, transitional cell carcinomas, ductal carcinomas, and squamous cell carcinomas.

As used herein the phrase “an individual of Ashkenazi descent” refers to a Jew of eastern or central European ancestry.

The method of determining predisposition to prostate cancer is effected by determining a presence or absence of at least one nucleic acid sequence alteration in at least one allele of a RNASEL gene of the individual, wherein the presence of the at least one nucleic acid sequence alteration indicates predisposition to prostate cancer in the individual.

The phrase “nucleic acid sequence alteration” refers to a mutation or single nucleotide polymorphism (SNP) including single nucleotide changes, or alterations of more than one nucleotide in a sequence, due to substitution, deletion, inversion or insertion in the wild type RNASEL nucleotide sequence.

Preferably, the sequence alteration in the RNASEL is:

(i) a deletion spanning nucleotides 471-474 of SEQ ID NO: 1;

(ii) a C to T substitution at nucleotide 354 of SEQ ID NO: 1; and/or

(iii) a deletion of G at nucleotide 11338427 of SEQ ID NO: 2.

To determine sequence alterations in the RNASEL gene, DNA is first obtained from a biological sample of the tested subject. Such biological samples include, but are not limited to, body fluids such as whole blood, serum, plasma, cerebrospinal fluid, urine, lymph fluids, and various external secretions of the respiratory, intestinal and genitourinary tracts, tears, saliva, milk as well as white blood cells, malignant tissues, amniotic fluid and chorionic villi.

Once the sample is obtained, DNA is extracted using methods which are well known in the art, involving tissue mincing, cell lysis, protein extraction and DNA precipitation using 2 to 3 volumes of 100% ethanol, rinsing in 70% ethanol, pelleting, drying and resuspension in water or any other suitable buffer (e.g., Tris-EDTA). Preferably, following such procedure, DNA concentration is determined such as by measuring the optical density (OD) of the sample at 260 nm (wherein 1 unit OD=50 μg/ml DNA).

To determine the presence of proteins in the DNA solution, the OD 260/OD 280 ratio is determined. Preferably, only DNA preparations having an OD 260/OD 280 ratio between 1.8 and 2 are used in the following procedures described hereinbelow.

The DNA sample is preferably amplified prior to determining sequence alterations, since many genotyping methods require amplification of the DNA region carrying the sequence alteration of interest.

In any case, once DNA is obtained, determining the presence of a sequence alteration in the RNASEL gene is effected using methods which typically involve the use of oligonucleotides which specifically hybridize with the nucleic acid sequence alterations in the RNASEL gene, such as those described hereinabove.

The term “oligonucleotide” refers to a single stranded or double stranded oligomer or polymer of ribonucleic acid (RNA) or deoxyribonucleic acid (DNA) or mimetics thereof. This term includes oligonucleotides composed of naturally-occurring bases, sugars and covalent internucleoside linkages (e.g., backbone) as well as oligonucleotides having non-naturally-occurring portions which function similarly to respective naturally-occurring portions.

Oligonucleotides designed according to the teachings of the present invention can be generated according to any oligonucleotide synthesis method known in the art such as enzymatic synthesis or solid phase synthesis. Equipment and reagents for executing solid-phase synthesis are commercially available from, for example, Applied Biosystems. Any other means for such synthesis may also be employed; the actual synthesis of the oligonucleotides is well within the capabilities of one skilled in the art and can be accomplished via established methodologies as detailed in, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988) and “Oligonucleotide Synthesis” Gait, M. J., ed. (1984) utilizing solid phase chemistry, e.g. cyanoethyl phosphoramidite followed by deprotection, desalting and purification by for example, an automated trityl-on method or HPLC.

The oligonucleotide of the present invention is of at least 17, at least 18, at least 19, at least 20, at least 22, at least 25, at least 30 or at least 40, bases specifically hybridizable with sequence alterations described hereinabove.

The oligonucleotides of the present invention may comprise heterocylic nucleosides consisting of purines and the pyrimidines bases, bonded in a 3′ to 5′ phosphodiester linkage.

Preferably used oligonucleotides are those modified in either backbone, intemucleoside linkages or bases, as is broadly described hereinunder.

Specific examples of preferred oligonucleotides useful according to this aspect of the present invention include oligonucleotides containing modified backbones or non-natural internucleoside linkages. Oligonucleotides having modified backbones include those that retain a phosphorus atom in the backbone, as disclosed in U.S. Pat. Nos.: 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050.

Preferred modified oligonucleotide backbones include, for example, phosphorothioates, chiral phosphorothioates, phosphorodithioates, phosphotriesters, aminoalkyl phosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms can also be used.

Alternatively, modified oligonucleotide backbones that do not include a phosphorus atom therein have backbones that are formed by short chain alkyl or cycloalkyl internucleoside linkages, mixed heteroatom and alkyl or cycloalkyl intemucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts, as disclosed in U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623, 070; 5,663,312; 5,633,360; 5,677,437; and 5,677,439.

Other oligonucleotides which can be used according to the present invention, are those modified in both sugar and the intemucleoside linkage, i.e., the backbone, of the nucleotide units are replaced with novel groups. The base units are maintained for complementation with the appropriate polynucleotide target. An example for such an oligonucleotide mimetic, includes peptide nucleic acid (PNA). A PNA oligonucleotide refers to an oligonucleotide where the sugar-backbone is replaced with an amide containing backbone, in particular an aminoethylglycine backbone. The bases are retained and are bound directly or indirectly to aza nitrogen atoms of the amide portion of the backbone. United States patents that teach the preparation of PNA compounds include, but are not limited to, U.S. Pat. Nos. 5,539,082; 5,714,331; and 5,719,262, each of which is herein incorporated by reference. Other backbone modifications, which can be used in the present invention are disclosed in U.S. Pat. No: 6,303,374.

Oligonucleotides of the present invention may also include base modifications or substitutions. As used herein, “unmodified” or “natural” bases include the purine bases adenine (A) and guanine (G), and the pyrimidine bases thymine (T), cytosine (C) and uracil (U). Modified bases include but are not limited to other synthetic and natural bases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further bases include those disclosed in U.S. Pat. No: 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B. , ed., CRC Press, 1993. Such bases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. [Sanghvi Y S et al. (1993) Antisense Research and Applications, CRC Press, Boca Raton 276-278] and are presently preferred base substitutions, even more particularly when combined with 2′-O-methoxyethyl sugar modifications.

Preferred methods of detecting sequence alterations involve directly determining the identity of the nucleotide at the alteration site by a sequencing assay, an enzyme-based mismatch detection assay, or a hybridization assay. The following is a description of some preferred methods, which can be utilized by the present invention.

Sequencing Analysis

The isolated DNA is subjected to automated dideoxy terminator sequencing reactions using a dye-primer cycle sequencing protocol. A sequence analysis can allow the identification of a sequence alteration.

Microsequencing Analysis

This analysis can be effected by conducting microsequencing reactions on specific regions of the RNASEL gene which may be obtained by amplification reaction (PCR) such as mentioned hereinabove. Genomic or cDNA amplification products are then subjected to automated microsequencing reactions using ddNTPs (specific fluorescence for each ddNTP) and appropriate oligonucleotide microsequencing primer oligonucleotides which can hybridize just upstream of the alteration site of interest. Once specifically extended at the 3′ end by a DNA polymerase using a complementary fluorescent dideoxynucleotide analog (thermal cycling), the primer is precipitated to remove the unincorporated fluorescent ddNTPs. The reaction products in which fluorescent ddNTPs have been incorporated are then analyzed by electrophoresis on sequencing machines (e.g., ABI 377) to determine the identity of the incorporated base, thereby identifying the sequence alteration in the RNASEL gene of the present invention.

It will be appreciated that the extended primer may also be analyzed by MALDI-TOF Mass Spectrometry. In this case, the base at the alteration site is identified by the mass added onto the microsequencing primer [see Haff and Smirnov, (1997) Nucleic Acids Res. 25(18):3749-50].

Solid phase microsequencing reactions, which have been recently developed can be utilized as an alternative to the microsequencing approach described above. Solid phase microsequencing reactions employ oligonucleotide microsequencing primers or PCR-amplified products of the DNA fragment of interest which are immobilized. Immobilization can be carried out, for example, via an interaction between biotinylated DNA and streptavidin-coated microtitration wells or avidin-coated polystyrene particles.

In such solid phase microsequencing reactions, incorporated ddNTPs can either be radiolabeled [see Syvanen, (1994),] Clin Chim Acta 1994;226(2):225-236] or linked to fluorescein (see Livak and Hainer, (1994) Hum Mutat 1994;3(4):379-385]. The detection of radiolabeled ddNTPs can be achieved through scintillation-based techniques. The detection of fluorescein-linked ddNTPs can be based on the binding of antifluorescein antibody conjugated with alkaline phosphatase, followed by incubation with a chromogenic substrate (such asp-nitrophenyl phosphate).

Other reporter-detection conjugates include: ddNTP linked to dinitrophenyl (DNP) and anti-DNP alkaline phosphatase conjugate [see Haju et al., (1993) Clin Chem 39:2282-2287]; and biotinylated ddNTP and horseradish peroxidase-conjugated streptavidin with o-phenylenediamine as a substrate (see WO 92/15712).

A diagnostic kit based on fluorescein-linked ddNTP with antifluorescein antibody conjugated with alkaline phosphatase is commercially available from GamidaGen Ltd (PRONTO).

Other modifications of the microsequencing protocol are described by Nyren et al. (1993) Anal Biochem 208(1):171-175 and Pastinen et al.(1997) Genome Research 7:606-614.

Mismatch Detection Assays Based on Polymerases and Ligases

The “Oligonucleotide Ligation Assay” (OLA) uses two oligonucleotides, which are designed to be capable of hybridizing to abutting sequences of a single strand of a target molecules. One of the oligonucleotides is biotinylated, and the other is detectably labeled. If the precise complementary sequence is found in a target molecule, the oligonucleotides will hybridize such that their termini abut, and create a ligation substrate that can be captured and detected. OLA is capable of detecting single nucleotide polymorphisms and may be advantageously combined with PCR as described by Nickerson et al. (1990) Proc. Natl. Acad. Sci. U.S.A. 87:8923-8927. In this method, PCR is used to achieve the exponential amplification of target DNA, which is then detected using OLA.

Other amplification methods which are particularly suited for the detection of single nucleotide polymorphism include LCR (ligase chain reaction), Gap LCR (GLCR). LCR uses two pairs of probes to exponentially amplify a specific target. The sequences of each pair of oligonucleotides, is selected to permit the pair to hybridize to abutting sequences of the same strand of the target. Such hybridization forms a substrate for a template-dependant ligase. In accordance with the present invention, LCR can be performed with oligonucleotides having the proximal and distal sequences of the same strand of a biallelic marker site. In one embodiment, either oligonucleotide will be designed to include the alteration site. In such an embodiment, the reaction conditions are selected such that the oligonucleotides can be ligated together only if the target molecule either contains or lacks the specific nucleotide that is complementary to the site of alteration on the oligonucleotide. In an alternative embodiment, the oligonucleotides will not include the site of alteration, such that when they hybridize to the target molecule, a “gap” is created as described in WO 90/01069. This gap is then “filled” with complementary dNTPs (as mediated by DNA polymerase), or by an additional pair of oligonucleotides. Thus at the end of each cycle, each single strand has a complement capable of serving as a target during the next cycle and exponential allele-specific amplification of the desired sequence is obtained.

Ligase/Polymerase-mediated Genetic Bit Analysis™ is another method for determining the identity of a nucleotide at a preselected site in a nucleic acid molecule (WO 95/21271). This method involves the incorporation of a nucleoside triphosphate that is complementary to the nucleotide present at the preselected site onto the terminus of a primer molecule, and their subsequent ligation to a second oligonucleotide. The reaction is monitored by detecting a specific label attached to the reaction's solid phase or by detection in solution.

Hybridization Assay Methods

Hybridization based assays which allow the detection of single base alterations rely on the use of oligonucleotide which can be 10, 15, 20, or 30 to 100 nucleotides long preferably from 10 to 50, more preferably from 40 to 50 nucleotides. Typically, the oligonucleotide includes a central nucleotide complementary to a polymorphic site of the RNASEL gene and flanking nucleotide sequences spanning on each. side of the central nucleotide and substantially complementary to the nucleotide sequences of the RNASEL gene spanning on each side of the polymorphic site. Examples of oligonucleotides which can be used in hybridization assays identifying the sequence alterations in the RNASEL gene of the present invention are provided in table 1, below.

TABLE 1

| | | |
|---|---|---|
| Mutation | Olignucleotide sequence | SEQ ID NO: |

| | | |
|---|---|---|
| 471delAAAG | 5′ ATTTGAGGCGAAAGACGAGGATCAAGAGCGGCTGA 3′ | 35 |
| Control | 5′ AATTTGAGGCGAAAGACAAAGGAGGATCAAGAGCGGCTGA 3′ | 36 |
| (471delAAAG) | | |
| 354C > T | : 5′ CTTTCTAAAGGAGCAGATGTTAATGAGTGTGATTTTTATG 3′ | 37 |
| Control | 5′ CTTTCTAAAGGAGCAGATGTCAATGAGTGTGATTTTTATG 3′ | 38 |
| (354C > T) | | |
| IVS5 + 1delG | 5′ GAAAAGCATAAAAATAAGTATTGTTTTC 3′ | 39 |
| Control | 5′ GAAAAGCATAAAAAGTAAGTATTGTTTTC 3′ | 40 |
| (IVS5 + 1delG) | | |

Bolded characters designate sequence alterations.

Sequence alteration can be detected by hybridization of the oligonucleotide of the present invention to the template sequence under stringent hybridization reactions.

By way of example, hybridization of short nucleic acids (below 200 bp in length, e.g. 1740 bp in length) can be effected by the following hybridization protocols depending on the desired stringency; (i) hybridization solution of 6×SSC and 1% SDS or 3 M TMACI 0.01 M sodium phosphate (pH 6.8), 1 mM EDTA (PH 7.6), 0.5% SDS, 100 μg/ml denatured salmon sperm DNA and 0.1% nonfat dried milk, hybridization temperature of 1-1.5° C. below the Tₘ, final wash solution of 3 M TMACI, 0.01 M sodium phosphate (pH 6.8), 1 mM EDTA (pH 7.6), 0.5% SDS at 1-1.5° C. below the Tₘ; (ii) hybridization solution of 6×SSC and 0.1% SDS or 3 M TMACI, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature of 2-2.5° C. below the Tₘ, final wash solution of 3 M TMACI, 0.01 M sodium phosphate (pH 6.8), 1 mM EDTA (pH 7.6), 0.5% SDS at 1-1.5° C. below the Tₘ, final wash solution of 6×SSC, and final wash at 22° C.; (iii) hybridization solution of 6×SSC and 1% SDS or 3 M TMACI, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature.

The detection of hybrid duplexes can be carried out by a number of methods. Typically, hybridization duplexes are separated from unhybridized nucleic acids and the labels bound to the duplexes are then detected. Such labels refer to radioactive, fluorescent, biological or enzymatic tags or labels of standard use in the art. A label can be conjugated to either the oligonucleotide probes or the nucleic acids derived from the biological sample (target). For example, oligonucleotides of the present invention can be labeled subsequent to synthesis, by incorporating biotinylated dNTPs or rNTP, or some similar means (e.g., photo-cross-linking a psoralen derivative of biotin to RNAs), followed by addition of labeled streptavidin (e.g., phycoerythrin-conjugated streptavidin) or the equivalent. Alternatively, when fluorescently-labeled oligonucleotide probes are used, fluorescein, lissamine, phycoerythirin, rhodamine (Perkin Elmer Cetus), Cy2, Cy3, Cy3.5, Cy5, Cy5.5, Cy7, FluorX (Amersham) and others [e.g., Kricka et al. (1992), Academic Press San Diego, Calif.] can be attached to the oligonucleotides.

Traditional hybridization assays include PCR, RT-PCR, RNase protection, in-situ hybridization, primer extension, Southern blot, Northern Blot and dot blot analysis.

Those skilled in the art will appreciate that wash steps may be employed to wash away excess target DNA or probe as well as unbound conjugate. Further, standard heterogeneous assay formats are suitable for detecting the hybrids using the labels present on the oligonucleotide primers and probes.

Two recently developed assays allow hybridization-based allele discrimination with no need for separations or washes [see Landegren U. et al., (1998) Genome Research, 8:769-776]. The TaqMan assay takes advantage of the 5′ nuclease activity of Taq DNA polymerase to digest a DNA probe annealed specifically to the accumulating amplification product. TaqMan probes are labeled with a donor-acceptor dye pair that interacts via fluorescence energy transfer. C1 cleavage of the TaqMan probe by the advancing polymerase during amplification dissociates the donor dye from the quenching acceptor dye, greatly increasing the donor fluorescence. All reagents necessary to detect two allelic variants can be assembled at the beginning of the reaction and the results are monitored in real time [see Livak et al., 1995 Hum Mutat 3(4):379-385]. In an alternative homogeneous hybridization based procedure, molecular beacons are used for allele discriminations. Molecular beacons are hairpin-shaped oligonucleotide probes that report the presence of specific nucleic acids in homogeneous solutions. When they bind to their targets they undergo a conformational reorganization that restores the fluorescence of an internally quenched fluorophore (Tyagi et al., (1998) Nature Biotechnology. 16:49].

It will be appreciated that a variety of controls may be usefully employed to improve accuracy of hybridization assays. For instance, samples may be hybridized to an irrelevant probe and treated with RNAse A prior to hybridization, to assess false hybridization.

Hybridization to Oligonucleotide Arrays

The chip/array technology has already been applied with success in numerous cases. For example, the screening of mutations has been undertaken in the BRCA1 gene and in the protease gene of HIV-1 virus [see Hacia et al., (1996) Nat Genet 1996;14(4):441-447; Shoemaker et al., (1996) Nat Genet 1996;14(4):450456; Kozal et al., (1996) Nat Med 1996;2(7):753-759].

The nucleic acid sample which includes the candidate region to be analyzed is isolated, amplified and labeled with a reporter group. This reporter group can be a fluorescent group such as phycoerythrin. The labeled nucleic acid is then incubated with the probes immobilized on the chip using a fluidics station. For example, Manz et al. (1993) Adv in Chromatogr 1993; 33:1-66 describe the fabrication of fluidics devices and particularly microcapillary devices, in silicon and glass substrates.

Once the reaction is completed, the chip is inserted into a scanner and patterns of hybridization are detected. The hybridization data is collected, as a signal emitted from the reporter groups already incorporated into the nucleic acid, which is now bound to the probes attached to the chip. Probes that perfectly match a sequence of the nucleic acid sample generally produce stronger signals than those that have mismatches. Since the sequence and position of each probe immobilized on the chip is known, the identity of the nucleic acid hybridized to a given probe can be determined.

For single-nucleotide polymorphism analyses, sets of four oligonucleotide probes (one for each base type), preferably sets of two oligonucleotide probes (one for each base type of the biallelic marker) are generally designed that span each position of a portion of the candidate region found in the nucleic acid sample, differing only in the identity of the polymorphic base. The relative intensity of hybridization to each series of probes at a particular location allows the identification of the base corresponding to the polymorphic base of the probe.

It will be appreciated that the use of direct electric field control improves the determination of single base mutations (Nanogen). A positive field increases the transport rate of negatively charged nucleic acids and results in a 10-fold increase of the hybridization rates. Using this technique, single base pair mismatches are detected in less than 15 sec [see Sosnowski et al., (1997) Proc Natl Acad Sci USA 1997;94(4):1119-1123].

Integrated Systems

Another technique which may be used to analyze sequence alterations includes multicomponent integrated systems, which miniaturize and compartmentalize processes such as PCR and capillary electrophoresis reactions in a single functional device. An example of such technique is disclosed in U.S. Pat. No. 5,589,136, which describes the integration of PCR amplification and capillary electrophoresis in chips.

These systems comprise a pattern of microchannels designed onto a glass, silicon, quartz, or plastic wafer included on a microchip. The movements of the samples are controlled by electric, electro-osmotic or hydrostatic forces applied across different areas of the microchip to create functional microscopic valves and pumps with no moving parts. Varying the voltage controls the liquid flow at intersections between the micro-machined channels and changes the liquid flow rate for pumping across different sections of the microchip.

When identifying sequence alterations, a microfluidic system may integrate nucleic acid amplification, microsequencing, capillary electrophoresis and a detection method such as laser-induced fluorescence detection. In a first step, the DNA sample is amplified, preferably by PCR. The amplification product is then subjected to automated microsequencing reactions using ddNTPs (specific fluorescence for each ddNTP) and the appropriate oligonucleotide microsequencing primers which hybridize just upstream of the targeted polymorphic base. Once the extension at the 3′ end is completed, the primers are separated from the unincorporated fluorescent ddNTPs by capillary electrophoresis. The separation medium used in capillary electrophoresis can for example be polyacrylamide, polyethyleneglycol or dextran. The incorporated ddNTPs in the single-nucleotide primer extension products are identified by fluorescence detection. This microchip can be used to process 96 to 384 samples in parallel. It can use the typical four-color laser induced fluorescence detection of ddNTPs.

Other methods which can be used to determine sequence alterations include but are not limited to conventional dot blot analyzes, single strand confornational polymorphism analysis (SSCP) as described by Orita et al. (1989) Proc. Natl. Acad. Sci. U.S.A. 86:2776-2770, denaturing gradient gel electrophoresis (DGGE), heteroduplex analysis, mismatch cleavage detection, and other conventional techniques as described in Sheffield et al.(1991), White et al.(1992), Grompe et al.(1989 and 1993). Another method for determining nucleic acid sequence alteration employs a specialized exonuclease-resistant nucleotide derivative as described in U.S. Pat. No. 4,656,127.

It will be appreciated that when utilized along with automated equipment, the above described detection methods can be used to screen multiple samples for the RNASELalterations of the present invention both rapidly and easily.

Sequence alterations can also be determined at RNASEL protein level (GenBarnk Accession No. NP_{—}066956). While chromatography and electrophoretic methods are preferably used to detect large variations in RNASEL molecular weight, such as detection of the truncated RNASEL protein generated by the 471delAAAG sequence alteration which results in a truncated protein of 55 amino acids, immunodetection assays such as ELISA and western blot analysis, immunohistochemistry and the like, which may be effected using antibodies specific to RNASEL sequence alterations are preferably used to detect point mutations and subtle changes in molecular weight Such methods may be used to detect for example, a stretch of 13 unique amino acids (SEQ ID NO:41) which is derived from the intron of RNASEL resulting from the loss of splice site at exon intron 5 (see Example 5 of the Examples section). Alternatively, antibodies directed at the N-terminal portion of RNASEL may also detect the truncated form of RNASEL, described hereinabove (i.e., 471delAAAG polypeptide product).

Thus, the present invention also envisages the use of serum immunoglobulins, polyclonal antibodies or fragments thereof, (i.e., immunoreactive derivatives thereof), or monoclonal antibodies or fragments thereof. Monoclonal antibodies or purified fragments of the monoclonal antibodies having at least a portion of an antigen-binding region, including the fragments described hereinbelow, chimeric or humanized antibodies and complementarily determining regions (CDR).

The term “antibody” as used in this invention includes whole antibody molecules as well as functional fragments thereof, such as Fab, F(ab′)₂, and Fv that are capable of binding with antigenic portions of the target polypeptide. These functional antibody fragments constitute preferred embodiments of the present invention, and are defined as follows:

(1) Fab, the fragment which contains a monovalent antigen-binding fragment of an antibody molecule, can be produced by digestion of whole antibody with the enzyme papain to yield an intact light chain and a portion of one heavy chain;

(2) Fab′, the fragment of an antibody molecule that can be obtained by treating whole antibody with pepsin, followed by reduction, to yield an intact light chain and a portion of the heavy chain; two Fab′ fragments are obtained per antibody molecule;

(3) (Fab′)₂, the fragment of the antibody that can be obtained by treating whole antibody with the enzyme pepsin without subsequent reduction; F(ab′)₂ is a dimer of two Fab′ fragments held together by two disulfide bonds;

(4) Fv, defined as a genetically engineered fragment containing the variable region of the light chain and the variable region of the heavy chain expressed as two chains; and

(5) Single chain antibody (“SCA”), a genetically engineered molecule containing the variable region of the light chain and the variable region of the heavy chain, linked by a suitable polypeptide linker as a genetically fused single chain molecule as described in, for example, U.S. Pat. No. 4,946,778.

Methods of generating such antibody fragments are well known in the art. (See for example, Harlow and Lane, Antibodies: A Laboratory Manual, Cold Spring Harbor Laboratory, New York, 1988, incorporated herein by reference).

Preferably, prior to generation of antibodies, the immunizing agent (e.g., peptide or antigenic portions thereof) is selected having a high antigenicity index. It will be appreciated that antigenicity analysis may be effected on the immunizing peptide alone or in the context of the native polypeptide which includes the immunizing peptide (for further details see http://www.epitope-informatics.com/References.htm. Antigenicity analysis using the www.bioweb.pasteur.fr/seqanal/interfaces/antigenic.html uncovered an antigenic epitope which corresponds to amino acid coordinates 4-10 of SEQ ID NO: 41. Such an epitope is thus preferably used for immunization.

Purification of serum immunoglobulin antibodies (polyclonal antisera) or reactive portions thereof can be accomplished by a variety of methods known to those of skill in the art including, precipitation by ammonium sulfate or sodium sulfate followed by dialysis against saline, ion exchange chromatography, affinity or immunoaffinity chromatography as well as gel filtration, zone electrophoresis, etc. (see Goding in, Monoclonal Antibodies: Principles and Practice, 2nd ed., pp. 104-126, 1986, Orlando, Fla., Academic Press). Under normal physiological conditions antibodies are found in plasma and other body fluids and in the membrane of certain cells and are produced by lymphocytes of the type denoted B cells or their functional equivalent. Antibodies of the IgG class are made up of four polypeptide chains linked together by disulfide bonds. The four chains of intact IgG molecules are two identical heavy chains referred to as H-chains and two identical light chains referred to as L-chains. Additional classes include IgD, Ig E, IgA, IgM and related proteins.

Methods for the generation and selection of monoclonal antibodies are well known in the art, as summarized for example in reviews such as Tramontano and Schloeder, Methods in Enzymology 178, 551-568, 1989. In general antibodies may be generated using in vitro or in vivo systems. In general, a suitable host animal is immunized with the recombinant RNASEL of the present invention. Advantageously, the animal host used is a mouse of an inbred strain. Animals are typically immunized with a mixture comprising a solution of the recombinant RNASEL of the present invention in a physiologically acceptable vehicle, and any suitable adjuvant, which achieves an enhanced immune response to the immunogen. By way of example, the primary immunization conveniently may be accomplished with a mixture of a solution of the recombinant RNASEL of the present invention and Freund's complete adjuvant, said mixture being prepared in the form of a water in oil emulsion. Typically the immunization will be administered to the animals intramuscularly, intradermally, subcutaneously, intraperitoneally, into the footpads, or by any appropriate route of administration. The immunization schedule of the immunogen may be adapted as required, but customarily involves several subsequent or secondary immunizations using a milder adjuvant such as Freund's incomplete adjuvant. Antibody titers and specificity of binding to the RNASEL can be determined during the immunization schedule by any convenient method including by way of example radioimmunoassay, or enzyme linked immunosorbant assay, which is known as the ELISA assay. When suitable antibody titers are achieved, antibody-producing lymphocytes from the immunized animals are obtained, and these are cultured, selected and cloned, as is known in the art. Typically, lymphocytes may be obtained in large numbers from the spleens of immunized animals, but they may also be retrieved from the circulation, the lymph nodes or other lymphoid organs. Lymphocytes are then fused with any suitable myeloma cell line, to yield hybridomas, as is well known in the art. Alternatively, lymphocytes may also be stimulated to grow in culture, and may be immortalized by methods known in the art including the exposure of these lymphocytes to a virus, a chemical or a nucleic acid such as an oncogene, according to established protocols. After fusion, the hybridomas are cultured under suitable culture conditions, for example in multi-well plates, and the culture supernatants are screened to identify cultures containing antibodies that recognize the hapten of choice. Hybridomas that secrete antibodies that recognize the recombinant RNASEL of the present invention are cloned by limiting dilution and expanded, under appropriate culture conditions. Monoclonal antibodies are purified and characterized in terms of immunoglobulin type and binding affinnity.

Antibody fragments according to the present invention can be prepared by proteolytic hydrolysis of the antibody or by expression in E. coli or mammalian cells (e.g. Chinese hamster ovary cell culture or other protein expression systems) of DNA encoding the fragment.

Antibody fragments can be obtained by pepsin or papain digestion of whole antibodies by conventional methods. For example, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, in U.S. Pat. Nos. 4,036,945 and 4,331,647, and references contained therein, which patents are hereby incorporated by reference in their entirety (see also Porter, R. R., Biochem. J., 73: 119-126, 1959). Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of V_{H} and V_{L} chains. This association may be noncovalent, as described in Inbar et al. (Proc. Nat'l Acad. Sci. USA 69:2659-62, 1972). Alternatively, the variable chains can be linked by an intermolecular disulfide bond or cross-linked by chemicals such as glutaraldehyde. Preferably, the Fv fragments comprise V_{H} and V_{L} chains connected by a peptide linker. These single-chain antigen binding proteins (sFv) are prepared by constructing a structural gene comprising DNA sequences encoding the V_{H} and V_{L} domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by Whitlow and Filpula, Methods, 2: 97-105, 1991; Bird et al., Science 242:423426, 1988; Pack et al., Bio/Technology 11:1271-77, 1993; and Ladner et al., U.S. Pat. No. 4,946,778, all of which are hereby incorporated, by reference, in entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells (see, for example, Larrick and Fry Methods, 2: 106-10, 1991).

Humanized forms of non-human (e.g., murine) antibodies are chimeric molecules of immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues, which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source, which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581′ (1991)]. The techniques of Cole et al. and Boemer et al. are also available for the preparation of human monoclonal antibodies (Cole et al., Monoclonal Antibodies and Cancer Therapy, Alan R. Liss, p. 77 (1985) and Boemer et al., J. Immunol., 147(1):86-95 (1991)]. Similarly, human monoclonal antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10, 779-783 (1992); Lonberg et al., Nature 368 856-859 (1994); Morrison, Nature 368 812-13 (1994); Fishwild et al., Nature Biotechnology 14, 845-51 (1996); Neuberger, Nature Biotechnology 14, 826 (1996); Lonberg and Huszar, Intem. Rev. Inmunol. 13 65-93 (1995).

The oligonucleotides and antibodies of the present invention can be included in a diagnostic or a therapeutic kit. Thus, the oligonucleotides and/or antibodies can be packaged in a one or more containers with appropriate buffers and preservatives and used for diagnosis or for directing therapeutic treatment.

The oligonucleotides or antibodies can be each mixed in a single container or placed in individual containers. Preferably, the containers include a label. Suitable containers include, for example, bottles, vials, syringes, and test tubes. The containers may be formed from a variety of materials such as glass or plastic.

In addition, other additives such as stabilizers, buffers, blockers and the like may also be added.

The oligonucleotides and antibodies of such kits can also be attached to a solid support, such as beads, array substrate (e.g., chips) and the like and used for diagnostic purposes.

Oligonucleotides and antibodies included in kits or immobilized to substrates may be conjugated to a detectable label such as described hereinabove.

The kit can also include instructions for determining if the tested subject is suffering from, or is at risk of developing prostate syndrome.

In addition to diagnostic advances pioneered by the present invention, the identification of sequence alterations in the RNASEL gene, which are strongly associated with prostate cancer especially in individuals of Ashkenazi descent allows for the design of therapeutic agents, which can be used to treat RNASEL specific prostate cancer.

Thus, according to another aspect of the present invention there is provided a method of treating a subject having prostate cancer or a predisposition thereto.

As used herein the term “treating” refers to preventing, curing, reversing, attenuating, alleviating, minimizing, suppressing or halting the deleterious effects of prostate cancer.

The method according to this aspect of the present invention is effected by specifically downregulating, in the subject (e.g., a mammal such as a human), expression of a mutated RNASEL transcript having at least one sequence alteration (e.g., the deletions described hereinabove).

Preferably, the method is effected by providing to the subject a therapeutically effective amount of an oligonucleotide capable of specifically inactivating the mutated RNASEL transcript.

Gene expression may be modified by designing complementary (i.e., antisense) oligonucleotides to the open reading frame, 5′, 3′, or other regulatory regions of the gene encoding RNASEL. Preferably, the oligonucleotide is designed to hybridize with the altered RNASEL sequences, leaving intact wild-type sequences. Similarly, inhibition can be achieved using triple helix base-pairing which inhibits the binding of polymerases, transcription factors, or regulatory molecules [Gee et al. In: Huber and Carr (1994) Molecular and Immunologic Approaches, Futura Publishing, Mt. Kisco N.Y., pp. 163-177]. A complementary molecule may also be designed to block translation by preventing binding between ribosomes and mRNA.

For efficient in vivo inhibition of gene expression using antisense technology, the oligonucleotides of the present invention must fulfill the following requirements (i) sufficient specificity in binding the target RNASEL sequence; (ii) water solubility (iii) increased resistance to nuclease degradation; (iv) capability of penetration through the cell membrane, (v) low toxicity.

Antisense molecules are typically, “chimeric antisense molecules” which contain two or more chemically distinct regions, each made up of at least one nucleotide. These oligonucleotides typically contain at least one region wherein the oligonucleotide is modified so as to confer upon the oligonucleotide increased resistance to nuclease degradation, increased cellular uptake, and/or increased binding affinity for the target polynucleotide. Such modifications include but are not limited to the addition of lipid moieties such as a cholesterol moiety, cholic acid, a thioether, e.g., hexyl-S-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxycholesterol moiety, as disclosed in U.S. Pat. No. 6,303,374.

It is not necessary for all positions in a given oligonucleotide molecule to be uniformly modified, and in fact more than one of the aforementioned modifications may be incorporated in a single compound or even at a single nucleoside within an oligonucleotide.

An additional region of the oligonucleotide may serve as a substrate for enzymes capable of cleaving RNA:DNA or RNA:RNA hybrids. An example for such include RNase H, which is a cellular endonuclease which cleaves the RNA strand of an RNA:DNA duplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate deoxyoligonucleotides hybridizing to the same target region. Cleavage of the RNA target can be routinely detected by gel electrophoresis and, if necessary, associated nucleic acid hybridization techniques known in the art.

Chimeric antisense molecules of the present invention may be formed as composite structures of two or more oligonucleotides, modified oligonucleotides, as described above. Representative U.S. patents that teach the preparation of such hybrid structures include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein fully incorporated by reference.

Ribozymes, enzymatic RNA molecules, may also be used to catalyze the specific cleavage of RNA. The mechanism of ribozyme action involves sequence-specific hybridization of the ribozyme molecule to complementary target RNA followed by endonucleolytic cleavage at sites such as GUA, GUU, and GUC. Once such sites are identified, an oligonucleotide with the same sequence may be evaluated for secondary structural features which would render the oligonucleotide inoperable. The suitability of candidate targets may also be evaluated by testing their hybridization with complementary oligonucleotides using ribonuclease protection assays.

Alternatively, downregulation of gene expression may be achieved using small interfering duplex oligonucleotides [i.e., small interfering RNA (siRNA)], which direct sequence specific degradation of mRNA through the previously described mechanism of RNA interference (RNAi) [Hutvagner and Zamore (2002) Curr. Opin. Genetics and Development 12:225-232].

As used herein, the phrase “duplex oligonucleotide” refers to an oligonucleotide structure or mimetics thereof, which is formed by either a single self-complementary nucleic acid strand or by at least two complementary nucleic acid strands. The “duplex oligonucleotide” of the present invention can be composed of double-stranded RNA (dsRNA), a DNA-RNA hybrid, single-stranded RNA (ssRNA), isolated RNA (i.e., partially purified RNA, essentially pure RNA), synthetic RNA and recombinantly produced RNA.

Preferably, the RNASEL specific small interfering duplex oligonucleotide of the present invention is an oligoribonucleotide composed mainly of ribonucleic acids.

Instructions for generation of duplex oligonucleotides capable of mediating RNA interference are provided in www.ambion.com.

Another agent capable of downregulating RNASEL gene expression is a DNAzyme molecule capable of specifically cleaving an mRNA transcript or DNA sequence of the RNASEL. DNAzymes are single-stranded polynucleotides which are capable of cleaving both single and double stranded target sequences (Breaker, R. R. and Joyce, G. Chemistry and Biology 1995;2:655; Santoro, S. W. & Joyce, G. F. Proc. Natl, Acad. Sci. USA 1997;943:4262) A general model (the “10-23” model) for the DNAzyme has been proposed. “10-23” DNAzymes have a catalytic domain of 15 deoxyribonucleotides, flanked by two substrate-recognition domains of seven to nine deoxyribonucleotides each. This type of DNAzyme can effectively cleave its substrate RNA at purine:pyrimidine junctions (Santoro, S. W. & Joyce, G. F. Proc. Natl, Acad. Sci. USA 199; for rev of DNAzymes see Khachigian, L M [Curr Opin Mol Ther4:119-21 (2002)].

Examples of construction and amplification of synthetic, engineered DNAzymes recognizing single and double-stranded target cleavage sites have been disclosed in U.S. Pat. No. 6,326,174 to Joyce et al. DNAzymes of similar design directed against the human Urokinase receptor were recently observed to inhibit Urokinase receptor expression, and successfully inhibit colon cancer cell metastasis in vivo (Itoh et al , 20002, Abstract 409, Ann Meeting Am Soc Gen Ther www.asgt.org). In another application, DNAzymes complementary to bcr-abl oncogenes were successful in inhibiting the oncogenes expression in leukemia cells, and lessening relapse rates in autologous bone marrow transplant in cases of CML and ALL.

To increase the effectiveness of the treatment described hereinabove, especially in cases where the individual is homozygous for the targeted mutation downregulation of the RNASEL mutants of the present invention is accompanied by expression of wild-type RNASEL in the target cells such as by using gene therapy procedures as further described hereinbelow.

The oligonucleotide (i.e., active ingredient) of the present invention can be provided to the subject per se, or as part of a pharmaceutical composition where it is mixed with a pharmaceutically acceptable carrier.

As used herein a “pharmaceutical composition ” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the preparation accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases. One of the ingredients included in the pharmaceutically acceptable carrier can be for example polyethylene glycol (PEG), a biocompatible polymer with a wide range of solubility in both organic and aqueous media (Mutter et al. (1979).

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intravenous, inrtaperitoneal, intranasal, or intraocular injections.

Alternately, one may administer a preparation in a local rather than systemic manner, for example, via injection of the preparation directly into a specific region of a patient's body.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the invention may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The preparations described herein may be formulated for parenteral administration, e.g., by bolus injection or continuous infusion. Formulations for injection may be presented in unit dosage form, e.g., in ampoules or. in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The preparation of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of active ingredients effective to prevent, alleviate or ameliorate symptoms of disease or prolong the survival of the subject being treated.

Determination of a therapeutically effective amount is well within the capability of those skilled in the art.

For any preparation used in the methods of the invention, the therapeutically effective amount or dose can be estimated initially from in vitro assays. For example, a dose can be formulated in animal models and such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p.1).

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions including the preparation of the present invention formulated in a compatible pharmaceutical carrier may also be prepared, placed in an appropriate container, and labeled for treatment of an indicated condition.

Pharmaceutical compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert.

The oligonucleotides of the present invention can also be expressed from a nucleic acid construct (described hereinabove), which can be administered to the subject employing any suitable mode of administration, described hereinabove (e.g., in-vivo gene therapy). Such a nucleic acid construct is introduced into a target cell or cells via appropriate gene delivery vehicle/methods (transfection, transduction, homologous recombination, etc.) and an expression system as needed and then the modified cells are expanded in culture and returned to the subject (i.e., ex-vivo gene therapy).

Expression of duplex oligonucleotides is preferably effected via expression vectors specifically designed for such use. For example, the pSUPER™ including the polymerase-III H1-RNA gene promoter with a well defined start of transcription and a termination signal consisting of five thymidines in a row (T5) [Brummelkamp (2002) Science 296:550-53]. Another suitable siRNA expression vector encodes the sense and antisense siRNA under the regulation of separate polIII promoters [Miyagishi and Taira[(2002) Nature Biotech. 20:497-500]. The resultant siRNA includes 5 thymidine termination signal. Alternatively, oligonucleotide sequences can be placed under bi-directional promoters to produce both the sense and antisense transcripts from the same promoter construct, thus simplifying the construction of expression vectors and achieving an equal molar ratio of cellular sense and antisense sequences. Examples for bi-directional promoters are disclosed in U.S. pat. appl. Ser. No. 20020108142.

It will be appreciated that when duplex oligonucleotide are used, transfection reagents dedicated to siRNA transfer to mammalian cells are preferably employed. Examples for such include but are not limited to siPORT™ Amine (i.e., a polyamine mixture) and siPORT™ Lipid (i.e., a mixture of cationic and neutral lipids).

Accordingly, in cases where the duplex oligonucleotides of the present invention are introduced into a cell in which RNA interference (RNAi) does not normally occur, the factors needed to mediate RNAi are introduced into such a cell or the expression of the needed factors is induced, as disclosed in U.S. Pat. appl. Ser. No.: 20020086356.

It will be appreciated that treatment of subjects exhibiting mutated RNASEL transcripts may also be effected using a “knock in” strategy (see U.S. Pat. No. 6,265,632), wherein endogenous RNASEL sequence alterations are corrected using advanced gene therapy.

The present invention may also find use in related applications.

Interferons (IFNS) are a family of related cytokines that mediate a range of diverse functions including antiviral, antiproliferative, antitumor and immunomodulatory activities. The pleiotropic activities of IFNs are mediated primarily through the transcriptional regulation of many downstream effector genes. IFNs bind to their cognate receptors and initiate a signaling cascade, involving the JAK-family of tyrosine kinases and STAT-family of transcription factors, that leads to the transcriptional induction of a number of IFN-stimulated genes (ISGs). IFN actions are largely mediated by the proteins encoded by ISGs, the best studied of which include the dsRNA-activated protein kinase (PKR), the 2′-5′ oligoadenylate (2-5A) synthetases and 2-5A-dependent ribonuclease (RNaseL) encoded by RNASEL gene, and the Mx proteins (U.S. Pat. No. 6,331,396).

It is appreciated that the 2-5A synthetase may be regarded as receptors/sensors for the presence of viral dsRNA which may allow the cell to respond by initiating a host-defence mechanism resulting in activation of RNASEl. Accordingly mice lacking RNASEL show enhanced susceptibility to enephlomyocardititis virus [see Silverman (2003) Biochemistry 42:1805-1812 and references therein].

It is thus conceivable that mutated ISGs such as mutated RNASEL will not be able to mediate the pleiotropic activities of IFNs.

In light of the significant costs of interferon treatment and deleterious side effects it is highly desirable to determine sensitivity of a subject to interferon therapy prior to treatment.

Thus, according to yet another aspect of the present invention there is provided a method of determining sensitivity of a subject to prospective interferon therapy.

The method is effected by determining a presence or absence of at least one sequence alteration in the RNASEL gene, such as described hereinabove, wherein the presence or absence of the sequence alteration indicates poor sensitivity of the subject to the prospective interferon therapy.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Materials and Experimental Procedures

PRCA Patients and Controls

The first stage of the study included 123 unselected PRCA patients and 383 controls including 133 elderly individuals without personal history of cancer and 250 young women. The majority of the PRCA patients were from Sharett Institute of Oncology, Hadassah Hebrew University Hospital, Jerusalem, seen during the years 1991-1997 [Hubert, (1999) Am. J. Hum. Genet. 65:921-924], and the rest were from Carmel Medical Center, Haifa, and Tel Aviv Sourasky Medical Center, Tel Aviv, Israel. Median age at diagnosis, available for 118 of 133 PRCA patients, was 68 years. The elderly control group consisted of Ashkenazi individuals (83 men and 50 women) residing in homes for the elderly in Jerusalem area [Hubert, (1999) Am. J. Hum. Genet. 65:921-924]. Their median age was 74 years at the time of blood sampling. A second control group consisted of 150 unselected Ashkenazi and 100 non-Ashkenazi healthy women aged 20-45 years, who underwent routine prenatal genetic screening. The female control group was added because women are not at risk for PRCA or related disorders, and the distribution of RNASEL alleles among them should, therefore, represent the underlying population. All participating subjects signed written informed consent and have identified themselves as Jews of Ashkenazi or non-Ashkenazi origin. DNA samples were blinded and tested in an anonymous manner.

The second stage of the study included 366 samples from Jewish PRCA patients and additional 100 samples of young women control. The Jewish PRCA patients included 222 unselected Ashkenazi and 144 non-Ashkenazi. Patients of Ashkenazi origin with other types of cancer were tested for RNASEL sequence variations. These included 276 samples of breast cancer and 301 samples of colon cancer.

Mutation Screening using DHPLC Hetroduplex Analysis

DHPLC analysis of the entire coding sequence of the RNASEL gene was first performed on selected number of PRCA individuals with family history using reverse-transcription PCR (RT-PCR). RNA extracted from the patients' lymphocytes using an RNA extraction kit (Qiagen, Chatsworth, Calif., USA) was used for the RT-PCT reactions. Complementary DNA was synthesized from 1 μg total RNA using 100 pmoles oligo d(T)₁₂₋₁₈ (Roche Diagnostics GmbH, Mannheim, Germany), 500 μM each DNTP (Pharmacia, Uppsala, Sweden), 10 U RNasin (Promega, Madison, Wis., USA) and 100 U Superscript II reverse transcriptase (Gibco BRL, Gathersburg, Md., USA) in a total volume of 10 μl. The reaction was incubated at 42° C. for 1 hour and terminated by heating at 90° C. for 2 minutes. PCR of RNASEL was performed using 8 pairs of overlapping primes that covered the entire cDNA (see Tables 2 and 3 hereinbelow SEQ ID NOs: 3-9 and 23-30). PCR reactions were performed in a final volume of 25 μl containing 0.5 μl cDNA, 1×PCR buffer (Roche Diagnostics GmbH, Mannheim, Germany), 200 μM each dNTP, 0.2 μM each primer (Sigma-Genosys Ltd., Rehovot, Israel), 5% DMSO and 1 U FastStart Taq Polymerase (Roche), using a Biometra” PCR system (Biometra, GmbH Gottingen, Germany). Cycling conditions included an initial denaturation step at 95° C. for 6 min, followed by 5 cycle s at 95° C. for 30 sec, 59° C. for 30 sec and 72° C. for 30 sec, and 40 cycles at 95° C. for 30 sec, 55° C. for 30 sec and 72° C. for 30 sec, and a final extension step of 72° C. for 7 min.

TABLE 2
Primers for DHPLC analysis of the RNASEL gene

| Exon | | | Nucleotides | Product |
|---|---|---|---|---|
| (*) | Primer/SEQ ID NO: | Sequence | (**) | size bp |
| 1(a) | RN19F1 (F)/3 | AAGCTTCTTTGATTAAGTGC | −85 Ex1 ATG | 414 |
| | RN432R2 (R)/4 | AGTTTCAGCAGCTTCAGC | 312-329 | |
| 1(b) | RN389F3 (F)/5 | TTTATCCTCGCAGCGATTG | 268-286 | 382 |
| | RN770R4 (R)/6 | GCGTAATAGCCTCCACATCAC | 647-667 | |
| 1(c) | RN725F5 (F)/7 | CATGCTCTCCTGAGCTCTGAC | 602-622 | 334 |
| | RN1058R6 (R)/8 | CCTTCACAAGGGAATGGTC | 937-955 | |
| 1(d) | RN1012F7 (F)/9 | TCTTGTTATGACAGCGAG | 909-926 | 392 |
| | RN1403R8 (R)/10 | CACACACAAACAAGTGGC | 1283-1300 | |
| 1(e) | RN1360F9 (F)/11 | ATTCTATGGGAGTGAGAGCCAC | 1257-1277 | 325 |
| | RNE1R (R)/12 | CACATTTACTCTAGGCCTTTC | +80 Ex 1 | |
| 2 | RNE2F (F)/13 | CCTTCCACAGAGATGATG | −113 Ex 2 | 272 |
| | RNE2R (R)/14 | AGAGAACCCTGACTACTACATG | +73 Ex 2 | |
| 3 | RN3EF (F)/15 | CACTCCTTCCAGGGTTAC | −67 Ex 3 | 401 |
| | RNE3R (R)/16 | CACACAGCCAGTAAATCC | +129 Ex 3 | |
| 4 | RNE4F (F)/17 | AAGGAAAGGGAGGGATGGGATG | −54 Ex 4 | 297 |
| | RNE4R (R)/18 | GAGCCTCTGGTTTGCCACTG | +107 Ex 4 | |
| 5 | RNE5F (F)/19 | ATATTTTGCCTTTTGGTATG | −82 Ex 5 | 297 |
| | RNE5R (R)/20 | GTAGATATAAACTTAGAATTGG | +80 Ex 5 | |
| 6 | RNE6F (F)/21 | CATGCTGAACAATTTGTG | −30 Ex 6 | 418 |
| | RNE6R (R)/22 | TAAAGCTTATGGACTAGTGTA | 2407-2427 | |

(*) Exon 1 is divided into 5 overlapping fragments
(**) nucleotides are numbered according to the coding sequence starting from the ATG

TABLE 3
Primers for RT-PCR analysis of the RNASEL gene

| | | | | |
|---|---|---|---|---|
| | | | Nucleotides | Product size |
| Fragment | Primer/SEQ ID NO: | Sequence | (**) | bp |

| | |
|---|---|
| 1-4 | Primer sequence is as indicated above in Table 1a for exon 1, |
| | fragments a-d |

| | | | | |
|---|---|---|---|---|
| 5 | RN1360F9 (F)/23 | ATTCTATGGGAGTGAGAGCCAC | 1257-1277 | 377 |
| | RN1736R10 (R)/24 | GAGCTTTCAGATCCTCAAATG | 1613-1633 | |
| 6 | RN1693F11 (F)/25 | TGTGGTAAAGAAGGGAAGCATC | 1590-1610 | 403 |
| | RN2095R12 (R)/26 | AGAGAACCCTGACTACTACATG | 1972-1992 | |
| 7 | RN2052F13 (F)/27 | AAAGAGGCAATTTCTACCAG | 1949-1968 | 414 |
| | RN2465R14 (R)/28 | TGCCAGGGACTGACATATC | 2344-2362 | |
| 8 | RN2423F15 (F)/29 | TTGTGAGGGATGAGTTGCATAG | 2320-2340 | 498 |
| | RN2920R16 (R)/30 | CATCCAGTGCCCAGACTTATAC | 2797-2817 | |

(**) nucleotides are numbered according to the coding sequence starting from the ATG

Screening of the PRCA patients and controls was then performed on the entire coding region of RNASEL gene including exon-intron boundaries, using 10 pairs of primers (SEQ ID NOS: 3-22). The amplification conditions were the same as those described above for the RT-PCR analysis.

PCR products were analyzed by a DHPLC apparatus (WAVE, Transgenomics Inc., Omaha, Neb., USA) according to the manufacturer's instructions and as previously described [Gavert et al. (2002)]. The temperatures, in which heteroduplex detection occurred, was determined by Transgenomic Wavemaker 4.1 software and the Stanford DHPLC melting program (http://.insertions.stanford.edu/melt.htm), which analyze the melting profile of each specific DNA fragment. For detecting homozygous for the mutation (i.e., 471delAAAG) and the silent variant (i.e., 354C>T), PCR products which generated a homoduplex peak on the DHPLC initial screen were mixed 1:1 with a PCR product amplified from a normal control DNA, and analyzed by the DHPLC for heteroduplex formation as described above.

Sequence Analysis

All samples in which variant bands were detected by DHPLC analysis, as well as normal bands to be used as controls, were analyzed by sequencing using the same PCR forward and reverse primers. Subsequent PCR products were first cleaned using a PCR purification kit (Qiagen, Chatsworth, Calif., USA) and sequenced using the Big Dye Terminator Chemistry (Perlin Elner Applied Biosystems, Foster City, Calif., USA). Following purification, sequencing reactions were analyzed using an automated ABI Prism 310 Genetic Analyzer according to the manufacturer's instructions.

Genotyping Analysis

DNA samples harboring the 471delAAAG, and the 354C>T silent variant, in addition to samples from random noncarrier PRCA patients, were genotyped using D1S2818 and D1S158 (max. heterozygosity 0.69 and 0.89, respectively) dinucleotide markers flanking the RNASEL gene on 1q25. PCR amplification was performed as described above using fluorescently end-labeled forward primers as follows: D1S2818 forward sequence, 5′-GAAGTTGCAGTGAACAGAG-3′ (SEQ ID NO: 31), reverse sequence, 5′-ATGCTAAGTTGTGTTGACTC-3′ (SEQ ID NO: 32); D1S158 forward sequence, 5′-GCTTCTCCATATT -TATTCAC-3′ (SEQ ID NO: 33), reverse sequence, 5′-AAAGGGCTGCTATCTGAG-3 ′ SEQ ID NO: 34). Following amplification, the PCR products were mixed with formamide loading buffer containing ROX-400 size marker (PE ABI), denatured and analyzed by an ABI PRISM 310 Genetic Analyzer (PE ABI). The peak areas were calculated using GeneScan Analysis Software (PE ABI).

Tumor Dissection and Loss of Heterozygosity Analysis

Tissues blocks containing malignant and benign prostatic hyperplasia (BPH) foci were dissected out, paraffin-embedded into new blocks and cut into 5 μm thick sections using sterile conditions. LOH for the 471delAAAG mutation was then determined using DHPLC. Tumor and BPH DNA was extracted by Qiagen PET mini columns (Qiagen, Chatsworth, Calif., USA) and amplified using 471delAAAG flanking primers (SEQ ID NOS: 5 and 6), as described above. The expected 382 bp PCR products were denatured, allowed to reanneal at room temperature as described above, and analyzed by DHPLC followed by sequencing.

Statistical Analysis

Analyses were performed using the SPSS® Base 11.0 software package (SSPS, Chicago, Ill., USA), and the EpiInfo 2000 software (http://www.cdc.gov/epiinfo/). The Odds Ratio index and confidence interval around it were calculated as an estimation of risk among mutation carriers. Chi square and Fisher exact tests were used when appropriate to allocate significant difference in mutations and markers frequencies.

### Example 2

### Detection of the RNASEL 471delAAAGframeshift Mutation in PRCA Patients

Unlike BRCA1/2 in breast cancer [Struewing, (1995) Nat. Genet. 11:198-200] and APC I1307K in colon cancer [Laken, (1997) Supra], very little is known about susceptibility genes for prostate cancer in Jewish men. To examine whether germline mutations of HPC1/RNASEL are associated with PRCA in Jewish patients, the entire coding sequence of RNASEL in leukocytes RNA from 2 pairs of Ashkenazi brothers affected with PRCA was analysed.

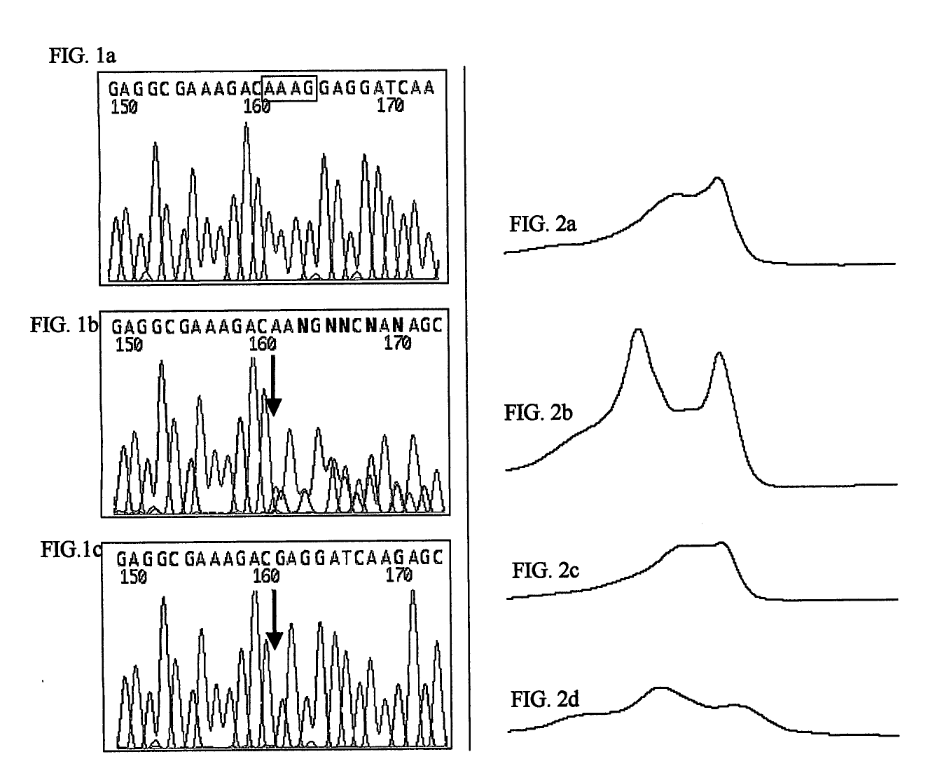

A novel, 4 base pair deletion mutation, 471delAAAG, was identified by sequencing (FIGS. 1a-c) and DHPLC (FIGS. 2a-c) in two brothers affected with PRCA (FIGS. 1b and 2b-c). The mutation starting from codon 157 in exon 1 results in a premature truncation at codon 164. The older brother which was diagnosed with PRCA at age 65 was found to be heterozygous for the 471delAAAG mutation while his younger brother, which was diagnosed at age 57 was homozygous for the same mutation.

### Example 3

### Frequency of RNASEL 471delAAAG Mutation and 354C>T Variant in Unselected PRCA Patients and Controls Stratified by Ethnic Origin

To assess the frequency of the 471delAAAG mutation and detect potentially new mutations in exon 1 of the RNASEL gene, DNA from additional unselected 119 PRCA patients (i.e., 85 Jews of Ashkenazi and 34 of non-Ashkenazi origin, respectively) and from 383 controls was analyzed using DHPLC.

As shown in Table 4 below, the 471delAAAG mutation was found in 15 out of 372 (4%) Ashkenazi and in none of the 134 non-Ashkenazi subjects tested (P≦0.025). In Ashkenazi PRCA patients, 471delAAAG was found in 7.9% (7/89, including 1 homozygote), significantly higher than the 1.5% carrier frequency detected in elderly population controls (2/133, OR=5.59; 95% CI: 1.1-27.5%, P≦0.025), and nearly significant if calculated for only the males elderly controls (2.4%, 2/83, OR=3.43; 95% CI 0.7-17.1, P≦0.2, Table 4 below). Among PRCA patients with a family history of cancer, the mutation was detected in 2 of 7 PRCA cases with a first-degree relative (28.6%) (OR=16.2; 95% CI 1.9-140.2, P≦50.01), and in a single case (3.4%, 1/29) with a family history of cancer. Analysis of germline DNA from 200 Ashkenazi women revealed 6 carriers among 150 young women (4%) and none in 50 elderly women (Table 4, below).

TABLE 4

| | | | | 471delAA |
|---|---|---|---|---|
| | No. | No. of carriers | | AG allele |
| Study groups | tested | (% of carriers) | | frequency |
| Mutation/Variant | | 471delAAAG | 354C > T | |
| Ashkenazi total | 372 | 15 (4.0%)^{a} | 14 (3.8%)^{b} | 0.022 |
| Unselected PRCA | 89 | 7 (7.9%)^{c,e,f} | 4 (4.5%)^{d,g} | 0.040^{i,j} |
| patients | | | | |
| First degree PRCA | 7 | 2 (28.6%)^{e,h} | None | 0.214 |
| affected | | | | |
| History of any cancer | 29 | 1 (3.4%) | 4 (13.8%) | 0.017 |
| in family | | | | |
| Elderly controls (total) | 133 | 2 (1.5%)^{c} | 6 (4.5%)^{d} | 0.008ⁱ |
| Males | 83 | 2 (2.4%)^{f,i} | 4 (4.8%)^{g} | 0.012^{j} |
| Females | 50 | None | 2 (4.0%) | 0^{k} |
| Young Females | 150 | 6 (4.0%) | 4 (2.7%) | 0.020^{k} |
| controls | | | | |
| Non-Ashkenazi total | 134 | None^{a} | None^{b} | 0 |
| Unselected PRCA | 34 | None | None | 0 |
| patients | | | | |
| Young females | 100 | None | None | 0 |
| controls | | | | |
| Total individuals | 506 | | | |
| tested | | | | |

Ashkenazi vs. non-Ashkenazi: P ≦ 0.025 for ^{a}471delAAAG, and for ^{b}354C > T.
Ashkenazi PRCA patients vs. total elderly controls:
^{c}for 471delAAAG, OR = 5.59 (95% CI 1.1-27.5; P ≦ 0.025) and
^{d}for 354C > T, OR = 0.99 (95% CI: 0.3-3.6).
^{e}Including one homozygous Individual.
Ashkenazi PRCA patients vs. elderly male controls:
^{f}for 471delAAAG, OR = 3.46 (95% CI: 0.7-17.1; P ≦ 0.2) and
^{g}for 354C > T, OR = 0.93 (95% CI: 0.23-3.8).
^{h}Patients with a first degree PPRCA affected relative vs. elderly male controls: OR = 16.2 (95% CI: 1.9-140.2; P ≦ 0.01)
471delAAAG allele frequency in Ashkenazi PRCA patients vs. ⁱtotal and ^{j}male elderly controls: P ≦ 0.01 and P ≦ 0.1, respectively.
^{k}471delAAAG allele frequency in Ashkenazi female controls: elderly vs. young: P ≦ 0.2.

An earlier disease onset (˜11 years) was shown to be associated with the RNASEL E265X mutation in Finnish families with PRCA [Rokman, (2002)]. In the group of Ashkenazi PRCA patients of the present invention, the median age of diagnosis was 64.5 years in 471delAAAG carriers, similar to that of non-carriers (68.5 years). However, it was 9.2 years less when compared to the median age of PRCA diagnosis (73.7 years) in Ashkenazi patients during 1997 (P≦0.001), as published by Israel National Cancer Registry, Ministry of Health [Registry, (1997)]

### Example 4

### RNASEL 471delAAAG Mutation is a Founder Mutation

DHPLC analysis of DNA samples from dissected tumor and benign prostatic hyperplasia (BPH) of a 471delAAAG carrier patient demonstrated loss of heterozygosity (LOH) in the tumor DNA in contrast with heterozygosity in the BPH smaple (FIG. 1c). Sequencing confirmed the sole presence of the 471delAAAG mutation allele in the tumor DNA. This is in agreement with the results of Carpten et al.[Carpten, (2002)] who showed LOH of the normal allele and absence of RNASEL protein in tumor cells from an HPC patient carrying the E265X mutation, further demonstrating the role of RNASEL in prostate cancer pathogenesis. It is of note that microsatellite analysis with the 2 flanking markers showed allelic heterozygosity in both the BPH and tumor DNA samples of our patient, suggesting that only the RNASEL gene was lost in this case. It is also noteworthy, that while null mutations of RNASEL are likely to contribute to PRCA tumorigenesis, homozygous truncating mutation in human and null mutation in mice [Zhou, (1997)] are compatible with life (i.e., not lethal).

Genotyping of two closely linked markers, D1S2818 and D1S158, flanking the RNASEL gene (approximately 2 Mb apart) revealed that the homozygous 471delAAAG patient was also homozygous for alleles containing 22- and 15-dinucleotide repeats, respectively. The D1S2818²²/D1S158¹⁵ haplotype was present in all (15/15) of 471delAAAG carriers compared to 32.5% (13/43) of non-carrier PRCA patients examined (P≦0.001). This finding strongly suggests that 471delAAAG is a founder mutation in the Ashkenazi population.

Interestingly, we found the 471delAAAG mutation in LNCaP cells, one of the most commonly used human prostate cancer cell lines, while the PC3 and DU145 cells did not carry this mutation. LNCAP cells originated from a lymphatic metastasis of a prostatic adenocarcinoma in a 50-year-old Caucasian male [Horoszewicz, (1980) Prog Clin Biol Res. 37:115-32], but it is not clear to us whether this patient was of Jewish Ashkenazi origin. The LNCaP cells, however, did not carry the commonly linked D1S158²² repeat allele. This is possibly due to many rearrangements encountered by the cells upon passages, or that this frameshift mutation occurred de novo in either this cell line, or in other Caucasian population.

### Example 5

### Detection of the RNASEL 354C>T and IVS5+1delG Allelic Variant

An additional allelic variant in RNASEL exon 1, 354C>T resulting in silent substitution (Val118), was identified in 14 of 372 Ashkenazi individuals (3.8%, Table 4, hereinabove and FIG. 2d) compared to none in 134 non-Ashkenazi tested (P≦0.025). This frequency was similar among PRCA patients and elderly controls. However, all 4 PRCA patients carrying this variant had family history of cancer. In 354C>T carriers, the most frequent flanking markers alleles were D1S158²¹ and D1S2818¹⁵, and their frequencies compared to noncarriers were 0.42 vs. 0.13 (P≦0.01) and 0.65 vs. 0.46 (P≦0.1), respectively. 85% of 354C>T carriers tested (11/13) carry both alleles compared to 17% (5/30) in noncarriers (P≦0.001), supporting a founder effect for this variant too. No individuals harboring both 471delAAAG and 354C>T genetic changes were detected.

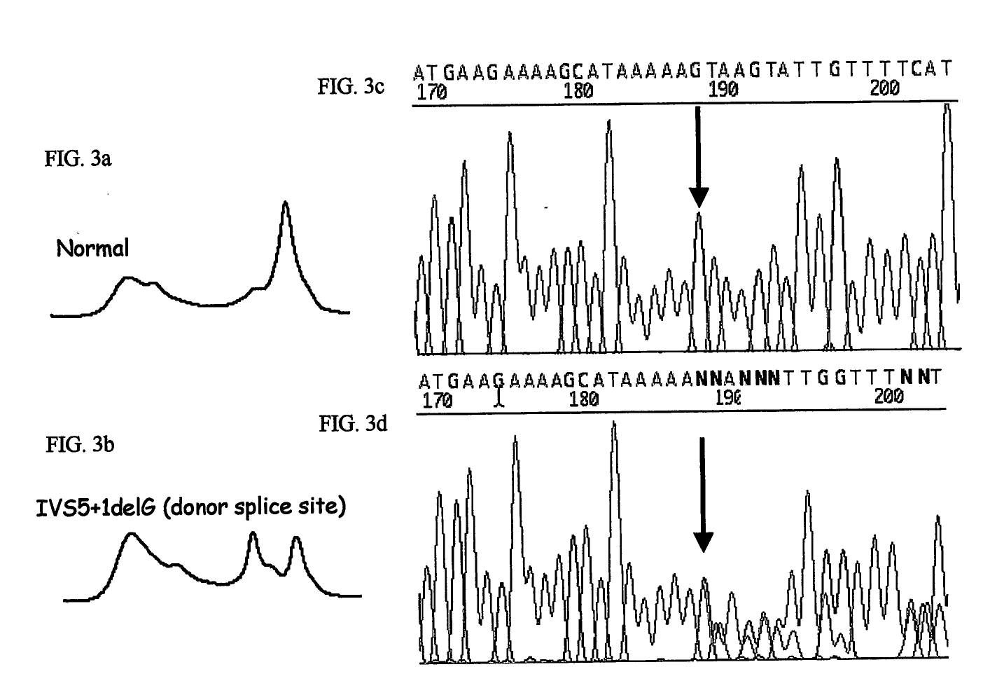

An additional allelic variant in RNASEL in exon-intron 5, IVS5+1delG, a donor splice site change, was identified in one Ashkenazi PRCA patient (FIGS. 3a-d). An RNASEL transcript including this mutation is predicted to encode a polypeptide of 692 amino acids including 679 amino acids of the wild-type sequence and 13 additional amino acids encoded from intron 5. This mutation is likely to cause a weak allele, further supporting the role of RNASEL in PRCA pathogenesis.

### Example 6

### RNASEL 471delAAAG in Ashkenazi Cancer Patients and Controls—A May 2003 Update

The 471delAAAG frameshift is the first founder RNASEL mutation detected in a specific population and the first germline mutation associated with an increased risk for PRCA in Ashkenazi Jewish men. 6 additional 471delAAAG carriers among 222 Ashkenazi PRCA patients were identified. Additional population based studies were done to determine the age-specific PRCA risk conferred by heterozygous and homozygous 471delAAAG mutations, and their possible association with familial clustering of PRCA or other cancers. Since RNASEL is present in many tissues, it was important to study whether 471delAAAG germline mutation is also associated with other cancers in Ashkenazi Jews. Currently, there is no significant difference in the frequency of the RNASEL 471delAAAG allele in those with breast cancer or colon cancer, compared to the controls. Table 5 below, summarizes the frequency of RNASEL 471delAAAGmutation in Ashkenazi cancer patients and controls. The frequency of RNASEL 471delAAAG mutation was higher in PRCA patients (3.9%) compared to the controls (2.4%) but it is not significant (OR=2.0, 95% CI 0.86-4.7; p=0.15), suggesting, that this mutation might be specific for sub-population of the Ashkenazi prostate cancer patients. The relatively high frequency of this cancer-associated mutation in the Ashkenazi population, combined with the molecular evidence regarding RNASEL role in PRCA pathogenesis, should prompt a cautious genetic counseling and diagnosis for individuals with PRCA and family members at high risk for the disease.

TABLE 5

| Study groups | No. Tested | No. Carriers (%) |
|---|---|---|
| Cancer Patients (probands): | | |
| PRCA (AJHG 2002) | 87 | 6 (6.9%)^{a} |
| | | P < 0.05 |
| PRCA (additional cases) | 222 | 6 (2.7%)^{b} |
| Total PRCA | 309 | 12 (3.9%)^{c} |
| Breast & Ovarian | 276 | 4 (1.5%) |
| Colon Cancer | 301 | 7 (2.3%) |
| Total Cancer Patients | 886 | 23 (2.6%) |
| Controls: | | |
| Elder males | 83 | 2 (2.4%) |
| Young females (20-40 years) | 250 | 7 (2.8%) |
| Females (45-80 years) | 177 | 1 (0.6%) |
| Total Controls | 510 | 10 (1.96%)^{a,b,c} |
| Total Ashkenazi Jews studied | 1396 | 33 (2.4%) |

Ashkenazi PRCA patients versus total controls:
^{a}OR = 3.7 (95% CI 1.3-10.5; p = 0.023)
^{b}OR = 1.4 (95% CI 0.5-3.9; p = 0.72)
^{c}OR = 2.0 (95% CI 0.86-4.7; p = 0.15)

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A method of determining predisposition of an individual of Ashkenazi descent to prostate cancer, the method comprising determining a presence or absence of at least one nucleic acid sequence alteration in at least one allele of a RNASEL gene of the individual, wherein said presence of said at least one nucleic acid sequence alteration indicates predisposition to prostate cancer in the individual.

2. The method of claim 1, wherein said at least one nucleic acid sequence alteration is selected from the group consisting of:
(i) a deletion spanning nucleotides 471-474 of SEQ ID NO: 1;
(ii) a C to T substitution at nucleotide 354 of SEQ ID NO: 1; and
(iii) a deletion at nucleotide 11338427 of SEQ ID NO: 2.

3. The method of claim 1, wherein said presence or absence of said at least one nucleic acid sequence alteration is determined in samples isolated from blood, malignant tissue, amniotic fluid, or chorionic villi.

4. The method of claim 1, wherein determining said presence or absence of said at least one nucleic acid sequence alteration is effected by the use of oligonucleotide hybridization.

5. The method of claim 1, wherein determining said presence or absence of said at least one nucleic acid sequence alteration is effected by an assay selected from the group consisting of PCR, DNA sequencing and SSCP analysis.

6. A method of determining predisposition of a subject to prostate cancer, the method comprising determining a presence or absence of at least one nucleic acid sequence alteration in at least one allele of a RNASEL gene of an individual, said at least one nucleic acid sequence alteration being selected from the group consisting of:
(i) a deletion spanning nucleotides 471-474 of SEQ ID NO: 1;
(ii) a C to T substitution at nucleotide 354 of SEQ ID NO: 1; and
(iii) a deletion at nucleotide 11338427 of SEQ ID NO: 2,
wherein said presence of said at least one nucleic acid sequence alteration indicates predisposition to prostate cancer in the individual.

7. An oligonucleotide specifically hybridizable with a nucleic acid sequence alteration selected from the group consisting of:
(i) a deletion spanning nucleotides 471-474 of SEQ ID NO: 1;
(ii) a C to T substitution at nucleotide 354 of SEQ ID NO: 1; and
(iii) a deletion at nucleotide 11338427 of SEQ ID NO: 2.

8. The oligonucleotide of claim 7, wherein said oligonucleotide is hybridizable with SEQ ID NO: 1 under hybridization conditions of hybridization solution containing 10% dextran sulfate, 1 M NaCl, 1% SDS and 5×10⁶ cpm ³²p labeled probe, at 65° C., with a final wash solution of 1×SSC and 0.1% SDS and final wash at 50° C.

9. The oligonucleotide of claim 7, wherein the oligonucleotide includes at least 10 nucleotides and no more than 50 nucleotides.

10. A kit for diagnosing prostate cancer or a predisposition to prostate cancer in a subject, the kit comprising the oligonucleotide of claim 7 and at least one reagent for detecting hybridization of the oligonucleotide with a nucleic acid sequence isolated from said subject.

11. The kit of claim 10, wherein said at least one reagent is selected suitable for detecting hybridization via an assay selected from the group consisting of PCR, RT-PCR, chip hybridization, RNase protection, in-situ hybridization, primer extension, Southern blot, Northern blot and dot blot analysis.

12. A method of treating a subject having, or being predisposed to, prostate cancer, the method comprising specifically downregulating in the subject expression of a mutated RNASEL transcript having at least one sequence alteration selected from the group consisting of:
(i) a deletion spanning nucleotides 471-474 of SEQ ID NO: 1;
(ii) a C to T substitution at nucleotide 354 of SEQ ID NO: 1; and
(iii) a deletion at nucleotide 11338427 of SEQ ID NO: 2, thereby preventing the formation, or halting the progression of, prostate cancer in the subject.

13. The method of claim 12, wherein specifically downregulating expression of said mutated RNASEL in the subject is effected by administering to the subject an oligonucleotide capable of specifically inactivating said mutated RNASEL transcripts.

14. The method of claim 13, wherein said oligonucleotide is a single or double stranded polynucleotide.

15. The method of claim 13, wherein said oligonucleotide is at least 10 nucleotides long.

16. The method of claim 13, wherein said oligonucleotide is hybridizable in either sense or antisense orientation.

17. A method of determining sensitivity of a subject to prospective interferon therapy, the method comprises determining a presence or absence of at least one nucleic acid sequence alteration selected from the group consisting of a deletion spanning nucleotides 471-474 of SEQ ID NO: 1 and/or a deletion at nucleotide 11338427 of SEQ ID NO: 2, wherein said presence of said at least one sequence alteration indicates poor sensitivity of the subject to the prospective interferon therapy.

18. The method of claim 17, wherein said presence or absence of said at least one nucleic acid sequence alteration is determined in samples isolated from blood, amniotic fluid, or chorionic villi.

19. The method of claim 17, wherein determining said presence or absence of said at least one nucleic acid sequence alteration is effected by the use of oligonucleotide hybridization.

20. The method of claim 17, wherein determining said presence or absence of said at least one nucleic acid sequence alteration is effected by an assay selected from the group consisting of PCR, DNA sequencing and SSCP analysis.

21. An antibody or antibody fragment being capable of specifically binding at least a portion of amino acid residues 1-55 of an RNASEL polypeptide.

22. An antibody or antibody fragment being capable of specifically binding a polypeptide including an amino acid sequence set forth in SEQ ID NO: 41.

23. The antibody or antibody fragment of claim 22, wherein the antibody or antibody fragment is directed at said amino acid sequence set forth in SEQ ID NO: 41.

24. A kit for diagnosing prostate cancer or a predisposition to prostate cancer in a subject, the kit comprising the antibody or antibody fragment of claim 22 and at least one reagent for detecting binding of the antibody or antibody fragment to a polypeptide isolated from said subject.

25. The kit of claim 24, wherein detecting binding of the antibody or antibody fragment to said polypeptide is effected by an assay selected from the group consisting of immunohistochemistry, ELISA, RIA, Western blot analysis, FACS analysis, an immunofluorescence assay, and a light emission immunoassay.

26. The kit of claim 24, wherein said antibody or antibody fragment is coupled to an enzyme.

27. The kit of claim 24, wherein said antibody or antibody fragment is coupled to a detectable moiety selected from the group consisting of a chromogem nc moiety, a fluorogenic moiety, a radioactive moiety and a light-emitting moiety.

28. A kit for diagnosing prostate cancer or a predisposition to prostate cancer in a subject, the kit comprising the antibody or antibody fragment of claim 23 and at least one reagent for detecting binding of the antibody or antibody fragment to a polypeptide isolated from said subject.

29. The kit of claim 28, wherein detecting binding of the antibody or antibody fragment to said polypeptide is effected by an assay selected from the group consisting of immunohistochemistry, ELISA, RIA, Western blot analysis, FACS analysis, an immunofluorescence assay, and a light emission immunoassay.

30. The kit of claim 28, wherein said antibody or antibody fragment is coupled to an enzyme.

31. The kit of claim 28, wherein said antibody or antibody fragment is coupled to a detectable moiety selected from the group consisting of a chromogenic moiety, a fluorogenic moiety, a radioactive moiety and a light-emitting moiety.

32. A method of determining predisposition of a subject to prostate cancer, the method comprising determining a presence or absence of at least one amino acid sequence alteration in an RNASEL polypeptide of an individual, said at least one amino acid sequence alteration being a translation product of:
(i) a deletion spanning nucleotides 471-474 of SEQ ID NO: 1; or
(ii) a deletion at nucleotide 11338427 of SEQ ID NO: 2,
wherein said presence of said at least one amino acid sequence alteration indicates predisposition to prostate cancer in the individual.


SEQUENCE LISTING (41 sequences)

SEQ ID NO: 1 - DNA, 4166 bp, Homo sapiens
caggtggaatgtcagaagactgagaacattgttccttcttcatactgctgctctgttgccagagaatcccaatttacactcaaagcttctttgattaagtgctaggagataaatttgcattttctcaaggaaaaggctaaaagtggtagcaggtggcatttaccgtcatggagagcagggatcataacaacccccaggagggacccacgtcctccagcggtagaagggctgcagtggaagacaatcacttgctgattaaagctgttcaaaacgaagatgttgacctggtccagcaattgctggaaggtggagccaatgttaatttccaggaagaggaagggggctggacacctctgcataacgcagtacaaatgagcagggaggacattgtggaacttctgcttcgtcatggtgctgaccctgttctgaggaagaagaatggggccacgccttttatcctcgcagcgattgcggggagcgtgaagctgctgaaacttttcctttctaaaggagcagatgtcaatgagtgtgatttttatggcttcacagccttcatggaagccgctgtgtatggtaaggtcaaagccctaaaattcctttataagagaggagcaaatgtgaatttgaggcgaaagacaaaggaggatcaagagcggctgaggaaaggaggggccacagctctcatggacgctgctgaaaaaggacacgtagaggtcttgaagattctccttgatgagatgggggcagatgtaaacgcctgtgacaatatgggcagaaatgccttgatccatgctctcctgagctctgacgatagtgatgtggaggctattacgcatctgctgctggaccatggggctgatgtcaatgtgaggggagaaagagggaagactcccctgatcctggcagtggagaagaagcacttgggtttggtgcagaggcttctggagcaagagcacatagagattaatgacacagacagtgatggcaaaacagcactgctgcttgctgttgaactcaaactgaagaaaatcgccgagttgctgtgcaaacgtggagccagtacagattgtggggatcttgttatgacagcgaggcggaattatgaccattcccttgtgaaggttcttctctctcatggagccaaagaagattttcaccctcctgctgaagactggaagcctcagagctcacactggggggcagccctgaaggatctccacagaatataccgccctatgattggcaaactcaagttctttattgatgaaaaatacaaaattgctgatacttcagaaggaggcatctacctggggttctatgagaagcaagaagtagctgtgaagacgttctgtgagggcagcccacgtgcacagcgggaagtctcttgtctgcaaagcagccgagagaacagtcacttggtgacattctatgggagtgagagccacaggggccacttgtttgtgtgtgtcaccctctgtgagcagactctggaagcgtgtttggatgtgcacagaggggaagatgtggaaaatgaggaagatgaatttgcccgaaatgtcctgtcatctatatttaaggctgttcaagaactacacttgtcctgtggatacacccaccaggatctgcaaccacaaaacatcttaatagattctaagaaagctgctcacctggcagattttgataagagcatcaagtgggctggagatccacaggaagtcaagagagatctagaggaccttggacggctggtcctctatgtggtaaagaagggaagcatctcatttgaggatctgaaagctcaaagtaatgaagaggtggttcaactttctccagatgaggaaactaaggacctcattcatcgtctcttccatcctggggaacatgtgagggactgtctgagtgacctgctgggtcatcccttcttttggacttgggagagccgctataggacgcttcggaatgtgggaaatgaatccgacatcaaaacacgaaaatctgaaagtgagatcctcagactactgcaacctgggccttctgaacattccaaaagttttgacaagtggacgactaagattaatgaatgtgttatgaaaaaaatgaataagttttatgaaaaaagaggcaatttctaccagaacactgtgggtgatctgctaaagttcatccggaatttgggagaacacattgatgaagaaaagcataaaaagatgaaattaaaaattggagacccttccctgtattttcagaagacatttccagatctggtgatctatgtctacacaaaactacagaacacagaatatagaaagcatttcccccaaacccacagtccaaacaagcctcagtgtgatggagctggtggggccagtgggttggccagccctgggtgctgatggactgatttgctggagttcagggaactacttattagctgtagagtccttggcaaatcacaacattctgggccttttaactcaccaggttgcttgtgagggatgagttgcatagctgatatgtcagtccctggcatcgtgtattccatatgtctataacaaaagcaatatatacccagactacactagtccataagctttacccactaactgggaggacattctgctaagattccttttgtcaattgcaccaaaagaatgagtgccttgacccctaatgctgcatatgttacaattctctcacttaattttcccaatgatcttgcaaaacagggattatcatccccatttaagaactgaggaacctgagactcagagagtgtgagctactggcccaagattattcaatttatacctagcactttataaatttatgtggtgttattggtacctctcatttgggcaccttaaaacttaactatccttccagggctcttccagatgaggcccaaaacatatataggggttccaggaatctcattcattcattcagtatttattgagcatctagtataagtctgggcactggatgcatgaattccactccttccagaaccaactgcattggttttccatgaccttaaggcagtagttctcaactggggggcaattttgcactgaagagagcatttggcagagtctgaagaagtttttggtgtcacagctttgtggggagcatgctatggcatttagtgggtaaagaccagggatgctgccaaacctgccttgcacaggacagcccctgcaacaaagaattatccagacaaaaatatcaatggtgctgaggttgagaaaacctgacttaaggggctgggatgcttttgaactagcttaaggcccaggactgtggagtgtgtggaccaccccacagaggagggactcagatttatttactcttgctggatctgtagtgatggagttccttctggtgtcagccccacaggaggctcccaggcctccctcacttcccatacccagtctaggagctccttctggctcccaagcacccagagctttcctccgccttttagttttggttcctccactggaatgtaggctcctcacgggcgatggctgtcttttcttgactttgtatcttcactgccaagcaaaaagtctgccaagtgggaatgtttaataaatattcattgaataatgaatgaaccatcttcgtacatgaataataatactgtcttacgtttttctggtgctttataatgtatacattacatctgagtattttattttatttaattttcaaaacaatcctttaaggtcaacattgttatccctattttgctgatgaggaaactaaggttagaaacattttgatttcctctaggacgtatagctaggaagtgttactatcttgatttgaacaaattttctggtgctaagtctgatgttctttccatgaatcattgtggtggttgagatggagctttgtaatgggaataaaacagtaccttaggttctttctgaaaaggaggtatctagcaatggataaatagataccactgaatgaaattaaatgttgattaggaacaaatttaaggcttaaaaaatactttatgagcagcaagattgctttaacttttaaaatgaagctttggttctctgatttgtaatgagcacctggacatgtcaattaaaatgcccatttgtgaagcttactcaataaaactttaaattgtcaaaaaaaaaaaaaaaaaa

SEQ ID NO: 2 - DNA, 15620 bp, Homo sapiens
gacgcagcaggaggaaactgctcgggctgcaagcagtcttccaggctttgcggctgccaaaggaataattgagacgtctgagttgagcaggtaaggcaggcagaaggaggaaagctagaaaacctcgataagtcgatcctgaagaattcttgggctttggagactggagaggccaggggctggaggacttgggaagtgggaggggagctcccagcccttggctgcccggaaggatcttcctccagtgcccttccagagttctgtcagagtccacacgtcatttatccatgtcctcagtttctcaggcatctctttgcgacttactcaggtcgcacagagaaagtgaaattgaaagcaggattgtctgttggagtacttccgcaggctcgtctcttctctactctgatgcgtcactcagggcttccttgatatttcctctccttcccggctcctttgtttataccagcccacctcacatctgattgtaatggggacggtctacacaaacactttggttcaaagatttcattgcctggtgatcgtgtatgtgttgcacaattaattaaacttctaagaggctaatcaattaacctctgaggctttgaaactgtcagagtggtgagattgctgaaaaagggttgcttcgtgaattctatagcagatgcagttatttaaatattcaggtaacagtttcgggctgtctgatgattgaactggatctgtaattctcagatctgattgacagttggacgaaattgatagctgccagttgcctattcgtgtagtagctatatgcacatgtgtagggagaatagaattcactaagcaatttattttttcataacgggatataattagctctaacattcattgagtggctaatatttgcaacactccatactttaatgtactttacacacatgactgtatttatttttgtctccatttcaggaaatagaaaattgagattcaacaatccaaggttacaggaagtaatagtaaaaaattacacagccacaagctcaaacttagatctccctaaggtcagagtacaagctcttttcactactgcagatactccttgacttatgatggggttacatacagataaacccatcctaaattgaaaatattgtaggtcaaaagcacatttaatacacctaatctactgaacgtcatagcttatcctagcttaccttaaatgtgctcagaacacttacattaggctacagttgggcaaaatcatctagctcaaagcttattttataataaagtgttaaatgtctcatgtatttatcgaatgctatgctgaaataaaaaacagaatggttgtatgtgtacttgaagtgtggtttctactgaatgtttattgcttttccaccactgcaaagtcaaaaaatcataaatcaaagtcaaaaaacataagtcagatattgtctgtactttaaataggggcacagggttgtacaaagaggacatgcacagagtcagcaaacacagtgatcaaccttcctggtgacataagaaatgcaaagggaaaaaaagccaagaaatatatttttcttattgaatgagggttcttctttttttattgataaataattgatgtgcatattttcatggtcttttttttttttttttggaagccacctatctctggtaagaatatatttatatgacattgaaagcaatttgttaatataggttaagaatcctaaatacaatcatatcttttgaatagtcattaaattctttgaagtctatcatatgaaatctatcataagatctatcgaaagaaaataaactaaaataagctgaatttgctgcaacatcatttagattttttaaatttttaattttaaaaaaccctttaaaacttatttttgaaaaccactcagtaatagagaaatgaaacaactgtatgatttcatattacatagtcatgaaaaatgttttacaagactttgcaataacacaggaaatgacttatagtatggtattaaatgaaaggaagtaggatacagttacttacagagtataattccaaatgtcttaaaaaatatatagaaatacactaaaatattaacattggctgtttctgggcattgggactagagaaataattttcttattttgaatttacatatatttataatattctgtaatttaaaagttttataagaaataaatatatgtatttagaaaaaatgctaatataattaatttctctgattcaagtgttttctccctattgtaaaattacaaagcacctctcttcgttgccaggtggaatgtcagaagactgagaacattgttccttcttcatactgctgctctgttgccagagaatcccaatttacactcaaagcttctttgattaagtgctaggagataaatttgcattttctcaaggaaaaggctaaaagtggtagcaggtggcatttaccgtcatggagagcagggatcataacaacccccaggagggacccacgtcctccagcggtagaagggctgcagtggaagacaatcacttgctgattaaagctgttcaaaacgaagatgttgacctggtccagcaattgctggaaggtggagccaatgttaatttccaggaagaggaagggggctggacacctctgcataacgcagtacaaatgagcagggaggacattgtggaacttctgcttcgtcatggtgctgaccctgttctgaggaagaagaatggggccacgccttttatcctcgcagcgattgcggggagcgtgaagctgctgaaacttttcctttctaaaggagcagatgtcaatgagtgtgatttttatggcttcacagccttcatggaagccgctgtgtatggtaaggtcaaagccctaaaattcctttataagagaggagcaaatgtgaatttgaggcgaaagacaaaggaggatcaagagcggctgaggaaaggaggggccacagctctcatggacgctgctgaaaaaggacacgtagaggtcttgaagattctccttgatgagatgggggcagatgtaaacgcctgtgacaatatgggcagaaatgccttgatccatgctctcctgagctctgacgatagtgatgtggaggctattacgcatctgctgctggaccatggggctgatgtcaatgtgaggggagaaagagggaagactcccctgatcctggcagtggagaagaagcacttgggtttggtgcagaggcttctggagcaagagcacatagagattaatgacacagacagtgatggcaaaacagcactgctgcttgctgttgaactcaaactgaagaaaatcgccgagttgctgtgcaaacgtggagccagtacagattgtggggatcttgttatgacagcgaggcggaattatgaccattcccttgtgaaggttcttctctctcatggagccaaagaagattttcaccctcctgctgaagactggaagcctcagagctcacactggggggcagccctgaaggatctccacagaatataccgccctatgattggcaaactcaagttctttattgatgaaaaatacaaaattgctgatacttcagaaggaggcatctacctggggttctatgagaagcaagaagtagctgtgaagacgttctgtgagggcagcccacgtgcacagcgggaagtctcttgtctgcaaagcagccgagagaacagtcacttggtgacattctatgggagtgagagccacaggggccacttgtttgtgtgtgtcaccctctgtgagcagactctggaagcgtgtttggatgtgcacagaggggaagatgtggaaaatgaggaagatgaatttgcccgaaatgtcctgtcatctatatttaaggctgttcaagaactacacttgtcctgtggatacacccaccaggatctgcaaccacaaaacatcttaataggtgagtccccaatccccaattctctcttagaaattgtgggatctttgtttatgataggaaaagtttttcattgcagagaggaaaggcctagagtaaatgtggatgattcaatttgtggatcattattacaacaaattcataattacagtgcagtgtctcctgctaccaattataactgggttctgttagtctacatgaaggttggtagatgccaacagaataccaggctagccttagattgccattggcaaaataaacagatgaaggcatgtcatggatcaaagtagctccagaaatctgagaatcctagaggtgagttccttcagacaaggtggcagtaaccatgaaacttactgacaaaattgaggtcaaagttgaggatgtcaggacaagatgtgacgtagtgaagacacagactccaaggcagaacaagtcagaatccactagtccaagccaaacaaagggaaggaaaatagcattttcttttaaatgttaaaagaaaagcaaggctgaccgtaaagtcccaggaagcaatatggtgcgggggagcaagccagggcttcagcgatgattagggtggcttgaagcctagtctcactagtttgtagttttgtaacctcagacaagtcacttgatactgtgagcctcagttcctcacttgaggctgataatgcctccttcgtaagtttgttatgaggtctaagtgcaatgatgaaatagtaagtcatttggtacatcataggtgctcaggaaaggctgttcttgcctgaaaggtctaagcagagtacataccttatccatagacatgcactgagatttaagctcagtgtaagggaacagctcctttacaaatctgtgctttcggcccctggaattgcatgaacgaggagctggttaaatttgtcctcatcccttcctaccccctggcctggaaaaaataaaatgtaagtgctgctcttggcagatacctaacagatcaaatgaaacagtgcacatggcagtgttttgaggggtgaatggcaaatacagatataaggtttattattgccttccacagagatgatgatttttatagcatgttacatatttatataaatcaattgactctcactttatggagaatgagtattttcaatgtgcctctgcaaacttttccttagattctaagaaagctgctcacctggcagattttgataagagcatcaagtgggctggagatccacaggaagtcaagagagatctagaggtaaattctacaatccagtttacctttcttctttctgcttccttattctttcatgtagtagtcagggttctctagagggacagaactagtaggacatatgtatatatgaaagggagtttattaaggagaattgactcacacggtcacaacataaagtcccataataagccatctgcaagttgaggaacaaggaagccagtgatgggtcagtctgagtaccaaaacctcaaaagtagggaagccgacagtgcagccttcagtctgtggccaaaggcctgaaagcccctggcaaaccactggtgtaagtccaaaactccataagctgaagaacttggatactgatgtttgagggcaggaagcatccagcacgggagaaagatgaaggccagaagtcttagcaagttgtctgctctttcattttctgcctgctttattttagccatgctggcagctgattagatggtgcccaccgtgactgaatgtgggtctgcctctcccagtccactgactcaaatgttgctctccttcggcaacaccctcacagacacacccagtcacaatactttgtatccttcaactcaatcaagttgacactcaatattaaccgtcacagttcattaccttatttcctatgggcaattggagggcattctattttaaccaggaaaaaaagaatgttcaaggctttccccatcttgctcactgtcctgactcccctgtaggccatggcatttccttgggcttctcccgcagcagagctttcaagatgacaatgggaaggtgcaacagcttgggtcggtgtggagttggctgtcactgtggacccttactgtatctctttgaatctctcttcctccactaccttaataagtaaataaactgagcatgccacatcttctctccaggagagaatttcaaatatctccaatattcattgaggagttatttttctacctaggaacaaagtaagtgaggtttctttcaccacaaactcaaactcgtgggctacactctgcagaaggctggatactaaggttctctgaaaactcctgggaggagtgaggtcaattcccagcttgaaagaacctaagcttggccaaaaagatgaggactctgaaatagctgttgctcctgaggggacatggagattggccgctgtagaccaaagcccccaggctgattctgaccctgtcagggtaggtctttcctggagagttttacagggtgctgcactttcaacccagtgggtgagactctccacccatatgtctggcctcctggtttggtggcatcagatttcagctccctccttgactggtggctagtgcaaagagttccttaggcgaccttcttcttggcctttcctcctggccacggcctaatccctggggtcatcactttgcctctcctggcctggactgacttttttatttgctgcgtgacatagcctcagacccaaagtcccagtttccagccaggctgtcagttctccctgtgtttaaagcattctccctttcctcaatttatctatattctgtccatacatttgaggcccagctcaaatctctcttcctccaagcagtctgccctaacaactgtgacttatatgtatcttttactcttcataaaactaagtggtatgggcgatatactgccttgaagaggtctgtatgcacacatctgtgccttccctccctaacaagcctacaaattccttgagattcctcccatcatcttgtttgctaatagcgtgcaccactccttccagggttacccaaataattagtaagcatctttggcttgatttatggcttttgtgcaggaccttggacggctggtcctctatgtggtaaagaagggaagcatctcatttgaggatctgaaagctcaaagtaatgaagaggtggttcaactttctccagatgaggaaactaaggacctcattcatcgtctcttccatcctggggaacatgtgagggactgtctgagtgacctgctgggtcatcccttcttttggacttgggagaggtaagtaaaactttgtgcagatgccacaggcctccaggatggaaagggtaaggtggtgcagacagaagtctgggctgcaaatcaggagacccaggcttttttcttagcttgggatttactggctgtgtgtccttggagagatgacttaacctctctggagtctgcttttcatttctgaagaataaggaggggaaaaaaagagatgcttgcctctcctatcatgcagaatttttgaagacatggttgtattacttttattaaaaaccaaaatctagccaggcgcggtggctcacgcctgtaatcccagcactttgggaggccgaggcgggtggatcatgaggtcaggagttccagaccagcctggtcagcatggtgaaaccccgtctctactaaaaatacaaaagttagccgagtgtggtggcaggtgcctgtaatgtcacctacttgggaagctgaggcaggagaattgcttgaacccgggaggtggaggttgcagtgagccaagattgcgctactgcactccagcttgggggacagagcgagactccatctcaaaaaaaaaaaaaaaaaaaaaaaaaagaaagaaaaaccaagaaagcacaaaaaacaccaaaatctaacggttttctttttctggaaaaggaaagggagggatgggatgatcatgatcatttagatcatttagtcatcttcagccgctataggacgcttcggaatgtgggaaatgaatccgacatcaaaacacgaaaatctgaaagtgagatcctcagactactgcaacctgggccttctgaacattccaaaagttttgacaagtggacgactaaggtatgaacaattcctatagtgcaggggttagtccaggaataagaatttgtgtaatgttttatgttagtttttctatcttattttacaccagtggcaaaccagaggctcaaagaggtgattttgcctcctgccctcccatccctatccccctaggctccctgccggggtgtctacttctactttcagaagctggccctccctctctcccattcagacccatgcccctctgatagactgttattcaggctccccctggatggccttatcccagcccatgagcctttccctcacttagctgataatcctattgccattatggcacgagcagctgactaaatagagcttaatccaagggaagtgcttcaccatgaggtgtttcagtccaggtacagcctggctgcctccactgcctgtaaacagcccagctctcaggatccacacacagacctacattctgcctgaggaaatagactatgaaggaaagatgatctttcaaattgctctctttaagaaaagttccacctaacttgcattaattatagtggaactctacaaaattagtcatcatctcaatctagtcagtactgtcagaactttcattattaattaacaaacatctatacaataccaattatgtgctaggcccatgcaaggccttgagcatacaaagcagaatacaatgtgtccctgtggggctgagaagcccatggaaatgatgctgtgggatgtgtgctgggcatcccagggacatgtgcaaagcactatgggagaacccataatcttcaggtcttggaaaccaacagcttcctatcctagacctgacctgggactctgcttctcctaattcttgttttgctttccttccctcactggtctctagactgcacatcacactcataattactggcttacggtccattttccttgctagactgtaagttccatgaaggcagagactacccattgtcttgtcagttattgtatccctggcatggagtagatgtgccatgactctttactaaatgaatgtctatcaaactttggctaaaattttaacttgtagaaacaaagaaatttgaaagcagctatgatagtgatagctaatgattacgtagtatcattatgattatgtggcagccatagttcttgagactttacatgagttaccacatttaatctttagcacaacttctgagacaatcatctatattttgcagatgagcaagctgaggcacagacaaataatttttccaactacacagaaccagtaaggggtgcaccaggatttgaagctgggaaggatggctgcagaggccatgtgcttaagccacaccactacatgaagctgtgatggcatcaagtctaccaactcatttactcactgaggaaattgaggctcagagaccacactcaaattactcatgctggacagtcagagattggcagacctggaaagaggaacttgttcttttatttcccaatttattggttcttttccctaaattccatgggcttatattgaattttgatagtcttcaggttttaacttcaagttttaccttttaattcccattttacactttgtttttctgttgacttgcccattttcatggtcacacaattaattttgtcagcataggtctggagcccaagttttccagcttgtatgactgtattggttcttgacttgccccatcctgccacacatacgtgcttggcgtttctcacgggtgttctgcacagagtgcacctggccagtctcagcagcatcctcactagcattgctctttcatcagttactcttcctaagtgacccactggattcttgactgttgcatcttgatctcgtagcccaaactctttctaaatatgagagccatctatggctctgtatctatgagggtgacaagcacagacaaagaggatgacattactgtgatatgaaaggagttgccctgggacacctgcaggcaaggagaagcaagtaaccttgtttaaaatataaattttagagggtactagtatagttttgttacatggatatattgtgtagtggtaaagttgggcttttagtgtaaccattacctgaataacgcacattgtacccattaagtaatttctcatccctctccctccaaccctcccacctttccaagtctccaattatattattccactctgtacatccatgtgtacacattattgagctcccacttataagagagaacatgtgatatttgactttctgtttgtaagtcatttcacttaaaataatggcctccagttccgtccatgtggccacaaaagacatgatttcattcttcttttttatggctgaaatggcccagacacattttctttatccaatcatctgttgatggacacttaggtggattccatatctttgctattgtgaatagtgctgtagtaaacatatgagtgccggtatctttttgatataagatttcttgggctgagtgcagtggctcacacctgtaatcccagcactttgggaggccgagatgggtggatcatttgaggtcaggagtttaagaccagcctggccaacatggcaaaaccctgtctctactaaaaatacaaaacttagccaagcattgtggcacacgtctgtaatcccagctacttgggcggctgaggcatgagaattgcttcagcctgtgaagcagaggttacagctttattttggcagatactcagtacattcttttctctgcatcctcatcaacatctgttactttttgactttttaatagtagcccctagtataagatgatatctcattggttttaatttgcatttctctgatgattagtgatgttaagcattttttcatatgctttttggcaatttgtacgtcttcttttgagaaatgtctattcttgtcatttgcccactttttaatggtgttattattttttattgttaagttgtttgagttccttgtaaattctggatatcaatcctctgttggatgcatagtttgcaaatattttttttcccatgctgcaggttgtttgttcactctgtttatttcttttgctgcgcagaagtttttagtttaattaaaccccatctgtttctttattttcattgcttgtgcttttggggtcttagtcatgaattctttgcctagaccaatgtccagaagagtttttcctaggttttcttttagtatttttatagtttcaggtcttacatttaagtctttaatccatcttgagttgatttttgtatacactgagagagagaggtctagttttaatcttctacatatggcagtctaattttcccagcaccacttactgaatagggtgtctttttcccagtgtatgtttttgccgactttgtctaagatcagttggttgtagatatgtggctttatttgtggcttctgtattctgttccattgatctatgtgtctatttttataccagtactgtgctgttttggttatcttagccttgcacaataattggaggtatatcataatgtgatgcctccaactttgtttttttttccttaagatgcctgtggctatttgggctcttttttggtttcatgtgaattttaggattttttctctaattctgtgaaaaatgatgttggtattttgatagggattgcattgaatctgtagattgctttgggcggggtgatcactttaacaatattagttattccttttggaaatatacctaaaaaaacaaaaacaaacaaacaaaaccgttcactttggccatggttgccaggcccagtttcagtctgaatagcccttttaaagaaagacatgtaatcaagtacgaatttgaaagggtggccacaggataaaaatattgtatagtataatgaacgactttcctagtatagtagtcaaagattttttaaccatattttaatatatttaatttcttatgaaaacaatttacttggaaaatacagacacacatttgaacatacttataatctcacatccagacacaattatatatatagttttaattttaaaatttttttaatttttttgccaggtgtggcggctcacacctgtaatcccagcactttgggggccaaggtgggtggatcacgaggtcaggagtttgagaccagcctggccaacatggtgaaaccctgtctctactaaaaatacaaaaaaaaaaaaattagctgggcatagtggcgggtgcctgtaatcccagctactcgggaggctgaggcaggagaatcgcttgaacccgggaggcggaggttgcagtgagccaagatcgcaccactgtactccagccctggtgacagagtgagactccgtcttgaaaaaaaaaaaattatttttttgagatggagtcttgctctgttactgagaccggagtgcagtggtgcgatctcagctctctgcaacctccacctcccaggttcaagtgactctcctgcctcagtctcccaagtagctgggattacaggcagcgccaccacgcctggctaattttttagcagaaacttttagtagaaacgaggtttcactgtgttggccaggctggtctcaatctcctgacctcaggtgatccgcccatttcggcctctgaaagtgctgggactacagatgtgagccaccccacccagcccagacacaattatagttagcattttggtatacagcatttacttccatttttaaattattttgctttattaattagttggtaattatagaatatatgaatatattcaaatttatattttgccttttggtatgaatatttcatcaggatttattttgtgttgctaccacatttttgtgttattttttgtttttttagattaatgaatgtgttatgaaaaaaatgaataagttttatgaaaaaagaggcaatttctaccagaacactgtgggtgatctgctaaagttcatccggaatttgggagaacacattgatgaagaaaagcataaaaagtaagtattgttttcattcctacaaattatatgtgaaatgagaacaatttaaaaaatccaattctaagtttatatctacgatggaaaaaagaaaatttattttgatgaaagaaaagtaattccttccctctgtgtgtaaaagggctatcactgttgccttacaattaatgagggacatgtccagcaggctttcatcttccttgtcatttttttgttggataaatggtaaaacaaattgaacaggagctagtgactcccttctgtgaagtaacacaggagactggacagctagataaagtgaaggtagagttatatacgtctaaaaatagcccacattagttagatcagcttttgctgacaaagatgttagttgcaaactcacaaaaaagccttgcagttcagggctcttctggattttggaattgtaggtagaggattgtggcattgcactatttccattttatgaaagaacaatctaaagttcagaaagattgtggccaaaggttgcacaattagttgaagagtcaggggaggagtcttgctctgtcagattccaaagtccttattcattctgcaatagaaatcaagcgagcaaagagatcttcagggccatatgctgtgaagcggggaagaataatttggggcaagcatgctgaacaatttgtgttttatttcaggatgaaattaaaaattggagacccttccctgtattttcagaagacatttccagatctggtgatctatgtctacacaaaactacagaacacagaatatagaaagcatttcccccaaacccacagtccaaacaagcctcagtgtgatggagctggtggggccagtgggttggccagccctgggtgctgatggactgatttgctggagttcagggaactacttattagctgtagagtccttggcaaatcacaacattctgggccttttaactcaccaggttgcttgtgagggatgagttgcatagctgatatgtcagtccctggcatcgtgtattccatatgtctataacaaaagcaatatatacccagactacactagtccataagctttacccactaactgggaggacattctgctaagattccttttgtcaattgcaccaaaagaatgagtgccttgacccctaatgctgcatatgttacaattctctcacttaattttcccaatgatcttgcaaaacagggattatcatccccatttaagaactgaggaacctgagactcagagagtgtgagctactggcccaagattattcaatttatacctagcactttataaatttatgtggtgttattggtacctctcatttgggcaccttaaaacttaactatccttccagggctcttccagatgaggcccaaaacatatataggggttccaggaatctcattcattcattcagtatttattgagcatctagtataagtctgggcactggatgcatgaattccactccttccagaaccaactgcattggttttccatgaccttaaggcagtagttctcaactggggggcaattttgcactgaagagagcatttggcagagtctgaagaagtttttggtgtcacagctttgtggggagcatgctatggcatttagtgggtaaagaccagggatgctgccaaacctgccttgcacaggacagcccctgcaacaaagaattatccagacaaaaatatcaatggtgctgaggttgagaaaacctgacttaaggggctgggatgcttttgaactagcttaaggcccaggactgtggagtgtgtggaccaccccacagaggagggactcagatttatttactcttgctggatctgtagtgatggagttccttctggtgtcagccccacaggaggctcccaggcctccctcacttcccatacccagtctaggagctccttctggctcccaagcacccagagctttcctccgccttttagttttggttcctccactggaatgtaggctcctcacgggcgatggctgtcttttcttgactttgtatcttcactgccaagcaaaaagtctgccaagtgggaatgtttaataaatattcattgaataatgaatgaaccatcttcgtacatgaataataatactgtcttacgtttttctggtgctttataatgtatacattacatctgagtattttattttatttaattttcaaaacaatcctttaaggtcaacattgttatccctattttgctgatgaggaaactaaggttagaaacattttgatttcctctaggacgtatagctaggaagtgttactatcttgatttgaacaaattttctggtgctaagtctgatgttctttccatgaatcattgtggtggttgagatggagctttgtaatgggaataaaacagtaccttaggttctttctgaaaaggaggtatctagcaatggataaatagataccactgaatgaaattaaatgttgattaggaacaaatttaaggcttaaaaaatactttatgagcagcaagattgctttaacttttaaaatgaagctttggttctctgatttgtaatgagcacctggacatgtcaattaaaatgcccatttgtgaagcttactcaataaaactttaaattgtc

SEQ ID NO: 3 - DNA, 20 bp, Artificial Sequence
  Single strand DNA oligonucleotide
aagcttctttgattaagtgc

SEQ ID NO: 4 - DNA, 18 bp, Artificial Sequence
  Single strand DNA oligonucleotide
agtttcagcagcttcagc

SEQ ID NO: 5 - DNA, 19 bp, Artificial Sequence
  Single strand DNA oligonucleotide
tttatcctcgcagcgattg

SEQ ID NO: 6 - DNA, 21 bp, Artificial Sequence
  Single strand DNA oligonucleotide
gcgtaatagcctccacatcac

SEQ ID NO: 7 - DNA, 21 bp, Artificial Sequence
  Single strand DNA oligonucleotide
catgctctcctgagctctgac

SEQ ID NO: 8 - DNA, 19 bp, Artificial Sequence
  Single strand DNA oligonucleotide
ccttcacaagggaatggtc

SEQ ID NO: 9 - DNA, 18 bp, Artificial Sequence
  Single strand DNA oligonucleotide
tcttgttatgacagcgag

SEQ ID NO: 10 - DNA, 18 bp, Artificial Sequence
  Single strand DNA oligonucleotide
cacacacaaacaagtggc

SEQ ID NO: 11 - DNA, 22 bp, Artificial Sequence
  Single strand DNA oligonucleotide
attctatgggagtgagagccac

SEQ ID NO: 12 - DNA, 21 bp, Artificial Sequence
  Single strand DNA oligonucleotide
cacatttactctaggcctttc

SEQ ID NO: 13 - DNA, 18 bp, Artificial Sequence
  Single strand DNA oligonucleotide
ccttccacagagatgatg

SEQ ID NO: 14 - DNA, 22 bp, Artificial Sequence
  Single strand DNA oligonucleotide
agagaaccctgactactacatg

SEQ ID NO: 15 - DNA, 18 bp, Artificial Sequence
  Single strand DNA oligonucleotide
cactccttccagggttac

SEQ ID NO: 16 - DNA, 18 bp, Artificial Sequence
  Single strand DNA oligonucleotide
cacacagccagtaaatcc

SEQ ID NO: 17 - DNA, 22 bp, Artificial Sequence
  Single strand DNA oligonucleotide
aaggaaagggagggatgggatg

SEQ ID NO: 18 - DNA, 20 bp, Artificial Sequence
  Single strand DNA oligonucleotide
gagcctctggtttgccactg

SEQ ID NO: 19 - DNA, 20 bp, Artificial Sequence
  Single strand DNA oligonucleotide
atattttgccttttggtatg

SEQ ID NO: 20 - DNA, 22 bp, Artificial Sequence
  Single strand DNA oligonucleotide
gtagatataaacttagaattgg

SEQ ID NO: 21 - DNA, 18 bp, Artificial Sequence
  Single strand DNA oligonucleotide
catgctgaacaatttgtg

SEQ ID NO: 22 - DNA, 21 bp, Artificial Sequence
  Single strand DNA oligonucleotide
taaagcttatggactagtgta

SEQ ID NO: 23 - DNA, 22 bp, Artificial Sequence
  Single strand DNA oligonucleotide
attctatgggagtgagagccac

SEQ ID NO: 24 - DNA, 21 bp, Artificial Sequence
  Single strand DNA oligonucleotide
gagctttcagatcctcaaatg

SEQ ID NO: 25 - DNA, 22 bp, Artificial Sequence
  Single strand DNA oligonucleotide
tgtggtaaagaagggaagcatc

SEQ ID NO: 26 - DNA, 22 bp, Artificial Sequence
  Single strand DNA oligonucleotide
agagaaccctgactactacatg

SEQ ID NO: 27 - DNA, 20 bp, Artificial Sequence
  Single strand DNA oligonucleotide
aaagaggcaatttctaccag

SEQ ID NO: 28 - DNA, 19 bp, Artificial Sequence
  Single strand DNA oligonucleotide
tgccagggactgacatatc

SEQ ID NO: 29 - DNA, 22 bp, Artificial Sequence
  Single strand DNA oligonucleotide
ttgtgagggatgagttgcatag

SEQ ID NO: 30 - DNA, 22 bp, Artificial Sequence
  Single strand DNA oligonucleotide
catccagtgcccagacttatac

SEQ ID NO: 31 - DNA, 19 bp, Artificial Sequence
  Single strand DNA oligonucleotide
gaagttgcagtgaacagag

SEQ ID NO: 32 - DNA, 20 bp, Artificial Sequence
  Single strand DNA oligonucleotide
atgctaagttgtgttgactc

SEQ ID NO: 33 - DNA, 20 bp, Artificial Sequence
  Single strand DNA oligonucleotide
gcttctccatatttattcac

SEQ ID NO: 34 - DNA, 18 bp, Artificial Sequence
  Single strand DNA oligonucleotide
aaagggctgctatctgag

SEQ ID NO: 35 - DNA, 35 bp, Artificial Sequence
  Single strand DNA oligonucleotide
atttgaggcgaaagacgaggatcaagagcggctga

SEQ ID NO: 36 - DNA, 40 bp, Artificial Sequence
  Single strand DNA oligonucleotide
aatttgaggcgaaagacaaaggaggatcaagagcggctga

SEQ ID NO: 37 - DNA, 40 bp, Artificial Sequence
  Single strand DNA oligonucleotide
ctttctaaaggagcagatgttaatgagtgtgatttttatg

SEQ ID NO: 38 - DNA, 40 bp, Artificial Sequence
  Single strand DNA oligonucleotide
ctttctaaaggagcagatgtcaatgagtgtgatttttatg

SEQ ID NO: 39 - DNA, 28 bp, Artificial Sequence
  Single strand DNA oligonucleotide
gaaaagcataaaaataagtattgttttc

SEQ ID NO: 40 - DNA, 29 bp, Artificial Sequence
  Single strand DNA oligonucleotide
gaaaagcataaaaagtaagtattgttttc

SEQ ID NO: 41 - protein, 13 aa, Artificial sequence
  Synthetic peptide
NKTCFHSTKLTVK